(19) United States
(12) Patent Application Publication
(10) Pub. No.: US 2014/0308294 A1
(43) Pub. Date: Oct. 16, 2014

(54) Anti-IL-1R1 Inhibitors For Use in Cancer

(71) Applicants: Anita Seshire, Rodgau (DE); Michael Wolf, Darmstadt (DE); Robert Tighe, North Reading, MA (US); Helen Sabzevari, Boston, MA (US)

(72) Inventors: Anita Seshire, Rodgau (DE); Michael Wolf, Darmstadt (DE); Robert Tighe, North Reading, MA (US); Helen Sabzevari, Boston, MA (US)

(73) Assignee: MERCK PATENT GMBH, Darmstadt (DE)

(21) Appl. No.: 14/110,173

(22) PCT Filed: Apr. 13, 2012
(86) PCT No.: PCT/US12/33490

§ 371 Date: Nov. 27, 2013

(30) Foreign Application Priority Data
Apr. 15, 2011 (EP) 11003183.8

(51) Int. Cl. C07K 16/28; C07K 14/715; G01N 33/574; C07K 16/30

(52) U.S. Cl. CPC C07K 16/2866; C07K 16/30; C07K 14/7155; G01N 33/57492
USPC 424/143.1; 530/389.1; 530/388.22; 530/350; 424/174.1; 514/19.3; 435/7.23

(57) ABSTRACT

The invention relates to polypeptides that block or inhibit the interleukin-1 receptor 1 (IL-1R1), the interaction of IL-1beta with IL-1R1 or the interaction between IL-1R1 and interleukin-1 receptor accessory protein (IL-1RaCP). The invention relates specifically to therapeutic polypeptides that target specifically IL-1R1 present on tumor cells, cancer stem cells, and cancer stem cells which are resistant to chemotherapy or radiotherapy. The invention specifically relates to cancer stem cells (CSC) that express IL-1R1 to which said inhibitors bind. Finally the invention relates to a combination therapy comprising killing tumorigenic differentiating cancer cells by means of standard chemo- or radiotherapy and prior or subsequent to that applying IL-1R1 inhibitors which target specifically CSC and strip the tumor of its capacity to generate cancer cell progeny.

### FIELD OF THE INVENTION

The invention relates to polypeptides that block or inhibit the interleukin-1 receptor 1 (IL-1R1), the interaction of IL-1beta with IL-1R1 or the interaction between IL-1R1 and interleukin-1 receptor accessory protein (IL-1RaCP). The invention relates specifically to therapeutic polypeptides that target specifically IL-1R1 present on tumor cells, cancer stem cells, and cancer stem cells which are resistant to chemotherapy or radiotherapy. The invention specifically relates to cancer stem cells (CSC) that express IL-1R1 to which said inhibitors bind. Finally the invention relates to a combination therapy comprising killing normally differentiating cancer cells by means of standard chemo- or radiotherapy, and prior or subsequent to that, applying IL-1R1 inhibitors which target specifically CSC.

### BACKGROUND OF THE INVENTION

Tumors are heterogenous in their cell composition. It is nowadays known, that not is every cell in a tumor is tumorigenic. Only a small subpopulation is able to reform new tumors. This cell population is able to self renew and to give rise to aberrant differentiated progeny. As these features are shared with stem cells the population was named cancer stem cells (CSC) [Reya, T., et al., Nature, 2001. 414(6859): p. 105-11], which are also referred to as tumor-initiating cells.

The concept that the bulk of cells that make up a tumor are derived from cancer stem cell (CSC) subpopulations has gained wide acceptance over recent years. CSCs are distinguished from the bulk population of tumor cells by their ability to successfully seed new tumors when implanted in low numbers into experimental animals, to do so reproducibly over several in vivo passages, and to recapitulate the morphology of the initial tumor. In contrast, the non-CSC population cannot initiate tumor growth in vivo even when implanted in high numbers. As the CSC subpopulation represents by definition the only tumor cells that are able to initiate the growth of new tumors, then CSCs should be expected to play a central role in metastasis formation.

CSC mostly comprise 1-10% of the tumor population depending on the context they are identified and on the tumor type. They are operationally defined by the following properties: (i) a selective capacity to initiate tumors and neoplastic proliferation, (ii) the ability for self-renewal, and (iii) the potential to give rise to more mature non-stem cell progeny by cell differentiation. Furthermore CSC are characterized by an increased resistance to chemo- and radiotherapy. Therefore, they are mainly quiescent or dormant and thereby circumvent conventional therapy regimen that target proliferating tumor cells. CSC are thought to be the source of metastasis as they actively migrate through the body, persist in osteoblastic niches in the bone marrow and home to various organs where they can reform new tumors [Trumpp, A. and O. D. Wiestler, Nat Clin Pract Oncol, 2008. 5(6): p. 337-47].

Homing is mainly mediated by surface molecules which allow these cells to migrate towards chemokine signal gradients. Therefore cytokine and chemokine signals as for instance Stromal-Derived-Factor (SDF)-1/Chemokine receptor (CXCR)-4, Osteopontin/CD44 or similar signals are thought to play an important role in CSC [Croker, A. K. and A. L. Allan, J Cell Mol Med, 2008. 12(2): p. 374-90]. As a logical is consequence the migration process through the body is only possible when these cells circumvent immune surveillance [Schatton, T and M. H. Frank, Ann N Y Acad Sci, 2009. 1176: p. 154-69]. Several cell surface markers has been identified on CSC in solid tumors, such as CD133+, CD44+, ABCB5+, whereas in hematological malignant tumors, such as AML and multiple myeloma above all CD34+, CD38+ were found.

The golden standard to define cancer stem cells is still given in vivo, when isolated or enriched cells are injected into immune-compromised mice and there form a new phenocopy of the original tumor. The original phenocopy contains the same amount of CSC as the original tumor though a slight enrichment can be propagated when tumors are serially re-transplanted. When grown without adherence in an ultra-low attachment (ULA) plate, without serum and under the addition of certain cytokines as EGF and/or bFGF CSC can form 3-dimensional (3D) structures that resemble spheroids formed by embryonic stem cells (ESC) and are called tumor spheres. Upon loss of adherence differentiated cells die by anoikis (detachment induced apoptosis) and therefore the tumor sphere assay enriches for CSC and immature progenitors with tumorigenic potential [Dontu, G. and M. S. Wicha, J Mammary Gland Biol Neoplasia, 2005. 10(1): p. 75-86]. After tumor spheres are formed, the compact aggregates can be dissociated by using enzymatic cocktails and further re-plated. Serial re-plating mimics the self renewal properties in vitro and give a hint on the stem cell capacities of the plated cells.

Conventional tumor therapy may initially shrink tumors by killing mainly tumor bulk populations with limited self-renewal and proliferative capacity, however according to the CSC hypothesis resistant CSC may remain viable after treatment and re-establish tumor growth leading to relapse and neoplastic disease progression.

In contrast to that, a novel therapy directed to and targeting CSC may reduce the tumors ability to generate cancer cell progeny, which inhibits tumor growth and might result in tumor degeneration. Preferred CSC targets and therapies would comprise those molecules or pathways that are preferentially induced or operative in malignant as opposed to physiological stem cells.

The effectiveness of cancer therapy is frequently impaired by either intrinsic or is acquired tumor resistance to cytotoxic agents or ionizing radiation.

Therefore, there is a need to develop a new and effective strategy to treat cancer diseases by involving CSC.

The IL-1 cytokine family consists of three members, IL1α (IL1A), IL1β (IL-1B) and IL1 receptor antagonist (IL1RA), each of which is encoded by a discrete gene. These genes give rise to precursor proteins that are proteolytically cleaved to give rise to the active cytokines. The two agonistic forms of the cytokine are IL-1α and IL-β. IL1α is mainly membrane bound and rarely found in the circulation whereas, conversely, IL-β is primarily secreted. IL-1α and IL-1β mediate the same downstream effects in IL-1 responsive cells. The third cytokine member, IL-1RA, fails to activate downstream signalling, and competitively inhibits the activity of IL-1α and IL-1β.

IL-1α, IL-1β and IL-1RA all mediate their effects by binding to the IL1 receptor type 1 (URI). Upon binding of IL-1α or IL-1β, IL1R1 associates with the IL1R3, also known as IL1 receptor accessory protein (IL1RAcP) resulting in the formation of an active signalling complex that induces a phosphorylation cascade involving IRAKs, MAPK p38, p42/44, ERK, JNK, STAT3 and activation of NF-κB. IL1R2 has been identified as a decoy receptor that does not promote intracellular signalling [1]. The magnitude of IL-1 responses is tightly regulated by the balance of antagonistic and agonistic players in the pathway.

The inflammatory response is a complex signalling cascade involving multiple cell types and cytokines. The inflammatory response to pathogens is usually initiated by macrophages, which upon their activation secrete IL-1R, which serves as an acute phase cytokine responsible for triggering the early physiological responses to infection. IL-1β sits at the apex of an inflammatory cascade that involves the to production of various additional cytokines and chemoattractants including IL8, IL6, MCP-1 and VEGF. Additionally, IL-1-driven Cox-2 activation leads to increased PGE² production. Under normal conditions the inflammation lasts until the infection is eradicated. If this reaction is impaired chronic inflammation can occur and is tightly linked to cancer.

As tumors progress, they accumulate a dense infiltrate of inflammatory cells that include tumor associated macrophages (TAM) and tumor infiltrating lymphocytes (TIL) as well as stromal fibroblasts and vascular endothelial cells. These cell types create a tumor microenvironment that is supportive of tumor growth, metastasis and neoangiogenesis. A major player in this orchestra is IL-1R.

The normal response to pathogens involves IL-1β activation by bacterial by-products like LPS, which is a strong inducer of IL-1β. The sensor cells for LPS are macrophages which secrete IL-1β. IL1R1 expressing cells like endothelial cells (ECs), epithelial cells, fibroblasts, chondrocytes and lymphocytes respond to IL-1β and start the secretion of pro-inflammatory mediators which are IL-1β itself, IL6, IL8, MCP-1, MKP-1 and Cox-2. To control this process and regulate homeostasis there are several physiological inhibitors of IL1R1, specifically the IL1 receptor antagonist and the IL1R2 receptor, which functions as a decoy receptor. Right panel. Pro-tumoral inflammation is a product of a complex tumor microenvironment, which consists of stromal fibroblasts, tumor associated macrophages (TAMS) and the tumor cells themselves. All 3 cell types secrete IL-1β and express IL-1α on their membrane. IL1-responsive cells are the tumor cells, TAMS, fibroblasts, ECs and lymphocytes. This results in the expression of various pro-inflammatory proteins like IL6, IL8, VEGF, MCP-1 and COX-2. Taken together these mediators support the pathogenesis of cancer by inducing STAT3 which supports tumor growth and immune suppression. Recruitment of neutrophils, COX-2 expression and MCP-1 also support immune suppression and VEGF drives tumor angiogenesis

Upon binding of IL-1β to IL1R1, IL1R1 heterodimerizes with IL1R accessory protein (IL1RaCP) and triggers a phosphorylation cascade involving IL1R associated Kinases (IRAK), MAPK, and ERK/JNK that culminates in the transcriptional activation of STAT3 and NF-κB. Thereby pro-inflammatory molecules are expressed and eventually secreted that inhibit anti-tumor immunity, promote tumor vascularization, and strengthen the tumor stroma.

Accordingly, the interaction of IL-1 with IL-1R1 has been implicated in the pathogenesis of several diseases, preferably immune and inflammatory diseases, such as arthritis (e.g., rheumatoid arthritis, osteoarthritis) and inflammatory bowel disease. Certain agents, including monoclonal antibodies, that bind IL-1R1 and neutralize its activity (e.g., IL-1Ra) have proven to be effective therapeutic agents for certain inflammatory conditions, such as coeliac disease, Crohn's disease; ulcerative colitis; idiopathic gastroparesis; pancreatitis, including chronic pancreatitis; acute pancreatitis, inflammatory bowel disease and ulcers, including gastric and duodenal ulcers, and moderately to severely active rheumatoid arthritis which can be treated with anti-IL-1R1 antibody AMG 108 (Amgen).

Other chimeric, humanized or human antibody directed to IL-1 alpha or beta (such as CDP-484, Celltech) or to the IL-1 receptor (for example, AMG-108, Amgen; R-1599, Roche), or IL-1Ra (anakinra, Amgen) are well known.

Most of these antibodies are used, as mentioned, in the treatment of inflammatory diseases. In some cases it is reported that such anti-IL-1R1 antibodies might be usable in treating lymphoproliferative disorders, including autoimmune lymphoproliferative syndrome (ALPS), chronic lymphoblastic leukemia, hairy cell leukemia, chronic lymphatic, leukemia, Burkitt's lymphoma, histiocytic lymphoma, and Hodgkin's disease. However, no evidence was demonstrated. Moreover, there are no reliable reports on the use of IL-1R1 inhibitors for the treatment of non-lymphoma derived cancer.

### SUMMARY OF THE INVENTION

The invention is based on the discovery that IL-1R1 is expressed or overexpressed on the surface of chemo-resistant and/or radio-resistant cancer cells, preferably cancer stem cells (CSC), whereas this receptor is not or only slightly expressed on normal differentiated proliferating non-tumorigenic tumor cells, or tumor cells that are not resistant to chemo- or radio-toxic agents, or are not CSC.

According to the invention, primary CSC from different cancer patients, such as non-small cell lung cancer (NSCLC) and colorectal carcinoma (CRC) patients, were analyzed in a specialized in vitro system (sphere assay) and compared them to CSC growing under differentiating conditions. In addition, the inventors selected resistant tumor cells from a NSCLC cell line by high dose chemotherapy treatment. They identified the cytokine IL1beta and its respective receptor as a differentially regulated gene. IL1beta is a cytokine that exhibit its signals on various cell types. In the context of a tumor it is produced by tumor cells and effects the tumor microenvironment by acting on endothelial cells, fibroblasts and infiltrating immune cells. Thereby it induces the expression of a number of proteins, including for instance matrix metallo-proteases, VEGF, bFGF, IL8, IL6 and others. The downstream processes create protection by disturbing immune surveillance and support tumor growth and metastasis. Inhibition of IL-1R1 by means of monoclonal antibodies or small chemical compounds can reduce growth of tumors (shown by using tumor spheres) which is associated with a reduced CSC phenotype. It is hereby stated that inhibition of IL-1R1 by therapeutic antibodies can be used to target suitable tumors and to treat respective tumor diseases.

The invention provides the following results:
1. Identification of IL1/IL1R1 signalling as a CSC-relevant pathway.
2. Expression of the IL-1β and IL1R1 genes is associated with decreased tumor-free and overall survival in cancer patients.
3. IL1R1 is expressed on primary CRC and NSCLC tumor cells and is upregulated following enrichment of CSC by serial tumor-sphere propagation.
4. IL1R1 is expressed on human primary tumor-derived CRC and NSCLC cell lines and its expression is upregulated in CSC-enriching tumor-spheres.
5. Antibody blockade of L1R1 inhibits tumor-sphere formation in vitro
6. IL1R1 blockade inhibits IL-1β-stimulated MAPKp38 and STAT3 phosphorylation.
7. Recombinant IL-16 induces the expression of IL1R1 in tumor-spheres, whereas antibody blockade of IL1R1 downregulates IL1R1 expression.
8. CSC-enriched tumor-spheres secrete the IL1-responsive cytokines hl L8 and hVEGF and production of these cytokines can be inhibited by IL1R1 blockade.
9. The IL1RA drug Kineret (Amgen) inhibits the growth of CSC-derived xenograft tumors & modulates serum cytokines in vivo.
10. Tumor associated macrophages (TAMS) promote tumor-sphere formation in is vitro through a mechanism involving IL-1

In summary and more generalizing, the invention relates to the following subject-matters:
An agent, preferably a polypeptide, more preferably a monoclonal antibody, that inhibits the interaction of IL1beta and IL1R1 for use in the treatment of cancer cells and/or cancer stem cells (CSC), preferably CSC in an individual.

The cancer cells according to the invention may comprise a subpopulation of cancer stem cells (CSC). The CSC according to the invention may comprise other tumor cells which are not CSC. The polypeptides according to the invention target IL1R1 preferably expressed on the surface of said CSC but not or not essentially on other tumor cells, which form the main population of the tumor tissue and are not cancer stem cells. Preferably, the cancer to be treated is resistant or mostly resistant to conventional chemotherapy and/or radiotherapy and/or other targeting therapies.
A respective agent, polypeptide or monoclonal antibody, that inhibits interaction of IL1beta and IL1R1 for use in the treatment of cancer cells and/or CSC, wherein the cancer to be treated is breast cancer, colorectal cancer (CRC) or non small cell lung cancer (NSCLC), preferably CRC.
A respective agent, polypeptide or a monoclonal antibody, that inhibits interaction of IL1beta and IL1R1 for use in the treatment of cancer cells and/or CSC, wherein the agent, preferably the antibody, is applied in combination with a cytostatic or cytotoxic agent or radiotherapy. Said cytostatic or cytotoxic agent is preferably an anti-tumor antibody such as herceptin, rituxan or erbitux, or a chemotherapeutic agent, which is applied to the individual prior or subsequent to said agent, preferably said monoclonal antibody, or simultaneously with said agent, preferably said antibody.
A pharmaceutical composition suitable for the treatment of cancer diseases comprising in a therapeutically effective amount an anti-IL-1R1 agent, preferably a polypeptide, more preferably an anti-IL-1R1 antibody as specified above together with a pharmaceutically acceptable excipient, diluent or carrier.
A pharmaceutical kit comprising at least a first and a second package, wherein (i) the first package comprises a respective anti-IL-1R1 agent, preferably a polypeptide or an anti-IL-1R1 antibody as described or a pharmaceutical composition comprising such an agent/antibody; and (ii) the second package comprises a cytotoxic and/or cytostatic agent or a pharmaceutical composition comprising said agent, wherein said second package is intended for administration prior or subsequent to the administration of the first package, preferably prior to said administration.
Use of an agent, preferably a polypeptide, more preferably a monoclonal antibody, that inhibits the interaction of IL1beta and IL1R1 for the manufacture of a medicament for the treatment of cancer cells and cancer tissue and/or cancer stem cells (CSC) alone or in conjunction with other tumor cells or tumor tissue which is not CSC or CSC tissue in an individual, wherein the cancer cells or tissue may comprise a subpopulation of cancer stem cells (CSC), and said CSC may comprise other tumor cells which are not CSC. Said polypeptide or antibody or fusion protein targets IL1R1 which is preferably exclusively expressed on the surface of said CSC but not or not essentially on other tumor cells, which form the main population of the tumor tissue and are not CSC. In a specific embodiment of the invention the cancer cells and/or CSC are widely resistant to conventional chemotherapy and/or radiotherapy and/or other targeting therapies.
The respective agent, polypetide, or monoclonal antibody, wherein said cancer is applied in combination with a cytostatic or cytotoxic agent or radiotherapy.
A method of treating a chemo- and/or radio-refractory cancer in an individual comprising administering to said individual an anti-IL-1R1 agent or antibody, preferably, wherein the chemo-, and/or radio refractory cancer was caused by a prior chemotherapy and/or radiotherapy in said individual.
The use of a polypeptide inhibiting the binding of IL1beta to IL1R1 and/or the heterodimerization of IL1R1 with IL1RaCP for the manufacture of a medicament for the treatment of a cancer cell population in an individual, wherein said cancer cell population comprises cancer stem cells (CSC) alone or together with other bulk tumor cells, and wherein optionally said CSC are resistant to standard chemotherapy and/or radiotherapy and/or standard targeting therapies (i).
The use of a polypeptide inhibiting the binding of IL1beta to IL1R1 and/or the heterodimerization of IL1R1 with IL1RaCP for the manufacture of a medicament for the treatment of cancer in an individual, wherein the cancer cell population comprises cancer stem cells (CSC), and optionally normal bulk tumor cells, wherein IL1R is expressed on the surface of said CSC but not or not essentially on non-CSC tumor cells (ii).
The use of a respective (i) (ii) polypeptide, wherein said cancer is selected from the group consisting of: colorectal cancer (CRC), non small cell lung cancer (NSCLC) and breast cancer (iii).
The use of a respective (i) (ii) (iii) polypeptide, wherein the polypeptide is administered to the individual in combination with a cytostatic agent, a cytotoxic agent, or radiotherapy, wherein, for example, the cytostatic or cytotoxic agent is an anti-tumor antibody, such as herceptin, rituxan or erbitux, or a chemotherapeutic agent, and wherein, for example, said polypeptide is applied to the individual prior to, simultaneously with, or after said cytostatic or said cytotoxic agent or said radiotherapy treatment.
The use of a polypeptide that inhibits or blocks the binding of IL1beta to IL1R1 and/or blocks or inhibits the heterodimerization of IL1R1 with IL1RaCP for the manufacture of a medicament in the treatment of cancer in an individual, wherein said cancer is colorectal cancer (CRC), non small cell lung cancer (NSCLC) or breast cancer, and wherein the polypeptide is targeting IL1R1 on CSC and/or other tumor cells, wherein IL1R1 is preferably expressed on the surface of CSC and not, or not essentially, or less than 50%, 60%, 70% or 80% (compared to the IL1R1 expression on CSC) on normal bulk tumor tissue cells (iv).
The use of a respective (iv) polypeptide for the manufacture of a medicament is for the treatment of cancer cells and/or CSC, wherein the CSC are resistant at least to standard chemotherapy and/or radiotherapy, optionally caused by prior treatment with chemotherapeutic, preferably cytotoxic and/or cytostatc agents (v).
The use of a respective (iv)(v) polypeptide for the manufacture of a medicament for the treatment of cancer cells and/or CSC, wherein the polypeptide is administered to the individual in combination with a cytostatic agent, or a cytotoxic agent, and/or in combination with radiotherapy (vi).
The use of a respective (i)-(vi) polypeptide, or a pharmaceutical composition comprising said polypeptide in a therapeutically effective amount, for the manufacture of a medicament for the treatment of cancer cells and/or CSC, wherein the polypeptide is selected from the group consisting of
(a) a murine, humanized, chimeric or human monoclonal antibody, preferably an anti-IL1R antibody, an anti-IL1beta antibody, an anti-IL1 aCP antibody (anti-IL-1 receptor accessory protein antibody), or a bispecific antibody targeting IL1R and IL1RaCP;
(b) a recombinant natural or modified IL1RA (interleukin-1 receptor antagonist),
(c) an IL1R1-IL1RaCP fusion protein acting as a trap for IL-1beta and preventing that IL-1beta is available for interaction with IL-1R1 (vii).
The use of a biomarker for assessing and predicting the effect of an agent on cancer in a cell based ex-vivo assay, wherein
(a) the biomarker is IL-1R1,
(b) the cells are chemo- or radio-resistant cancer stem cells (CSC) expressing IL1R1 on their surface,
(c) and said agent is a therapeutic polypeptide as specified under (i)-(vii), wherein, preferably, said CSCs were obtained as subpopulation of cells of to tumor tissue samples of an individual by treating these tissue samples with a chemotherapeutic agent and/or by radiation, and wherein further, in a preferred embodiment of the invention, the cancer cells in said cell based assay derive from samples of an individual suffering from NSCLC, CRC or breast cancer.

### DESCRIPTION OF THE FIGURES

FIG. 1: CSC-markers are enriched in high dose chemotherapy-selected cells.

A. ABC-transporters are known to be highly enriched in CSC. They are responsible for the rapid drug efflux which leads to resistance to common therapy regimen. Various ABC-transporters are up-regulated upon high-dose chemotherapy-selection of the A549 NSCLC cell line.
B. One further mechanism by which CSC gain resistance to common therapy regimen is the elevated expression of detoxifying enzymes as Aldehyde-dehydrogenases (ALDH). Also various isoforms of ALDH are up-regulated upon high-dose chemotherapy-selection.

FIG. 2: CSC-markers are up-regulated in re-plated tumor spheres from primary patient material derived from NSCLC and CRC patients.

A. The tumor sphere assay-enriches for CSC, in contrast adherent conditions mainly drive differentiation. Adherently passaged cells were analyzed vs. re-plated tumor spheres on a microarray, which revealed elevated expression of ABCG2 and ALDH-1, which are known CSC-markers.
B. Increased ALDH-1 expression was functionally shown with the Aldefluor assay. Increase of ˜10 folds in re-plated tumor spheres compared to the initially plated cells.
C. Increased ABCG2-expression increase the amount of cells in the side-population, Increase in SP-cells could be shown in re-plated tumor spheres. ˜10-fold increase compared to the initially plated cells.

FIG. 3: Identification of IL1beta and its respective receptor IL as differentially up-regulated targets in re-plated primary tumor spheres and chemotherapy-selected cells.

A. IL1beta is stable in adherent (differentiating) conditions, but up-regulated in tumor spheres from patient-derived, primary NSCLC cells.
B. IL1beta is significantly up-regulated in re-plated tumor spheres when compared to the adherent control. The expression of the respective receptor IL1R1 was also elevated in re-plated tumor spheres from patient-derived, primary NSCLC cells.
C. IL1beta expression is up-regulated in tumor spheres from patient-derived, primary CRC cells.
D. IL1beta expression is up-regulated in re-plated CRC tumor spheres when compared to the adherent control. The expression of the respective receptor IL1R1 was significantly elevated in re-plated tumor spheres from patient-derived, primary CRC cells.
E. IL1beta is significantly up-regulated in high-dose Paclitaxel (Pac) and Doxorubicin (Dox) selected cells.

FIG. 4: The IL is expressed on tumor cells and its expression in increased in replated tumor spheres.

A. Patient derived primary NSCLC cells were used to induce s.c. xenograft tumors in immune-compromised mice. After tumor induction single cells were isolated and further analyzed for target expression of URI. The CSC-marker CD133 is low abundant in these population.
B. Single cells from s.c. xenografts (A.) were re-plated in a tumor sphere assay. Expression of URI was increased by ˜7 folds and expression of the CSC-marker CD133 was increased by ˜15 folds. Therefore URI seems to be a CSC-associated surface molecule.

FIG. 5: Disease free and overall survival is correlated with IL1beta and IL expression levels in patients with lung adenocarcinoma stage I and CRC.

A. Probe set 215561_s_at for IL1beta shows a survival benefit in overall survival of patients with lung adenocarcinoma stage I and a benefit in disease free survival of CRC patients. Overall survival in CRC is not significantly correlated.
B. Probe set 39402_at for URA is slightly correlated with a better survival in lung adenocarcinoma stage I and is correlated with a survival benefit in disease free and overall survival in CRC.

FIG. 6: Inhibition of IL1R1 by a neutralizing antibody reduces tumor sphere formation in a dose dependent manner.

A.B. Patient derived primary cells were plated in a tumor sphere assay with either 0.5 and 10 μg/mL control IgG (normal goat IgG) or anti-human URI antibody. Untreated cells were used as a positive control for sphere-formation.
A. Anti-IL-1R1 treatment reduced tumor sphere formation in a dose dependent manner in primary NSCLC. B. A dose-dependent reduction of tumor sphere formation observed in primary CRC. The experiment was performed in triplicate, the bar graphs depict fold tumor-sphere induction vs. control; error bars represent SD.

FIG. 7: The IL1R1 is expressed on cell lines derived from primary CRC and NSCLC tumors and its expression is upregulated following plating as tumor-spheres. Patient derived primary NSCLC and CRC cells were cultivated under differentiating adherent conditions in vitro and stained for IL1R1 expression using a polyclonal or monoclonal antibody and expression was detected by flow cytometry. The same cell lines were also subjected to one round of culture as tumor-spheres. Expression of IL1R1 in the tumor-spheres was compared to the adherent cells as shown in the histogram overlays (adherent=red, spheres=blue) for NSCLC (upper panel) and CRC (lower panel). Both antibodies detected a substantial upregulation of IL1R1 in NSCLC sphere cultures vs. adherent cultures, whereas, in the CRC spheres, only the polyclonal antibody was able to detect URI upregulation.

FIG. 8: Detection of IL1R1 using two different antibodies in primary NSCLC and CRC.

Patient derived primary NSCLC and CRC cells were cultivated under differentiating adherent conditions in vitro and stained for IL1R1 expression using a functional grade PAB goat AF269 from R&D Systems labelled with APC and a MAB hIgG4 15C4 corresponding to an Amgen Mab from patent WO2004022718A labelled with APC. As a respective Isotype control goat-IgG1-APC was used and did not give any signal as shown with the blue line in the left and middle panels from up and down. The red line in the same panels shows URI target recognition by both antibodies. Results obtained with the two anti-IL1R1 antibodies is compared in the right panels (up and down) and shows that the polyclonal antibody AF269 (red histogram) has a stronger target recognition than the monoclonal 15C4 antibody (blue histogram), most probably due to recognition of multiple epitopes.

FIG. 9: Activation of pMAPKp38 and pSTAT3 by IL-1β and inhibitory effect of the 15C4 Mab on this pathway.

Patient derived primary NSCLC cells were plated as spheres and in absence of growth factors. After over night incubation at 37° C./5% CO² A.C: recombinant IL1 was added in increasing concentrations from 0.1 pg/mL to 100 pg/mL. Cells were incubated for 20 min and then lysed with HGNT-buffer. As a control adherently grown NSCLC cells were lysed assuming a differentiated phenotype. B.D. another portion of spheres was induced with 1 pg/mL recombinant IL-1β for 30 min and then increasing concentrations of the anti-IL1R1 hIgG4 15C4 Mab were added and incubated fpr 24h and then lysed fpr further analysis. A.B.C.D. Lysates were analyzed using a Pathscan ELISA for either pSTAT3 or pMAPKp38, respectively, following the manufacturers instructions. Results are shown as OD450 nm in arbitrary units. The results are preliminary and confirmation of the results is still pending.

FIG. 10: Detection of IL1R1 levels by western blot following stimulation with recombinant IL-1β or treatment with an anti-IL1R1 antibody.

Patient derived primary NSCLC and CRC tumor-spheres were plated overnight in the absence of growth factors at 37° C./5% CO² A.C: Recombinant IL-1β was added in increasing concentrations from 0.1 pg/mL to 100 pg/mL. Cells were incubated for 20 min and then lysed with HGNT-buffer. Lysates from adherently grown NSCLC, representing a differentiated phenotype, were used as a control. B.D: Tumor-spheres were stimulated with 1 pg/mL of recombinant IL-1β for 30 min followed by addition of increasing concentrations of the 15C4 anti-IL1R1 antibody. 24 h later, lysates from the cells were prepared. A.B.C.D: Lysates were analyzed via western blot. Membranes were blocked with 5% milk and incubated with an anti-IL1R1 rabbit IgG (Millipore) primary antibody at 1:1000 in TBS. Detection was made with an anti-rabbit-POD secondary antibody at 1:2000 in TBS. Membranes were imaged using a VersaDoc device. Densitometric analysis was performed with Quantity one software, the results of which are depicted as bar graphs in arbitrary units above. The results are preliminary and confirmation of the results is still pending.

FIG. 11: Secretion of IL1-responsive cytokines hIL8 and hVEGF in vitro can be blocked by an anti-IL1R1 antibody.

Patient derived primary NSCLC cells were plated as tumor-spheres and cultured in the absence of growth factors overnight at 37° C./5% CO² A.B: Supernatants of spheres and adherently grown cells were collected after 24 h and analyzed for hIL8 and hVEGF levels using quantitative ELISAs from R&D Systems. Results are shown as a bar graph with cytokine concentrations in pg/mL. C.D: Spheres were stimulated with 1 pg/mL recombinant IL-1β for 30 min followed by the addition of increasing concentrations of the 15C4 anti-IL1R1 Mab. After 6 h of incubation supernatants were collected and hIL8 and hVEGF levels were measured by ELISA. Results are shown as bar graph with cytokine concentrations in pg/mL The results are preliminary and confirmation of the results is still pending.

FIG. 12: Tumor growth inhibition of primary CRC and NSCLC-CSC-tumors using the IL1RA drug Kineret from Amgen.

CSC were FACS-enriched from primary tumor-derived NSCLC and CRC cell line cultures on the basis ALDH activity as determined by the Aldefluor assay. 10⁴ cells/mouse were transplanted s.c. in NOD/SCID mice together with Matrigel. A Loading dose of 5 μg/mL Kineret (recombinant URA, Amgen) was applied to the Matrigel and subsequent daily treatment s.c. with either 5 or 10 mg Kineret was performed beginning on day 1 post transplantation. Tumor volumes were monitored weekly and three mice from each group were sacrificed at day 76 for the CRC model (A.) and at day 91 for the NSCLC model (B.) for further analysis. For the remaining animals Kineret treatment was stopped and tumor progression was continually monitored. When tumors reached >2000 mm³ mice were sacrificed. At time of sacrifice serum was collected along for cytokine analysis. A. Tumor growth curves in the CRC tumor model study and B. growth curves in the NSLCL model study. Curves represent mean tumor volume in mm3 from 10 mice/group; error bars are SEM The data shown for each model is representative of n=2 similar experiments.

FIG. 13: Detection of hIL8 & hVEGF in the serum of NOD/SCID mice engrafted with CSC-xenograft tumors.

This analysis was performed on serum collected from the tumor model studies described in FIG. 18. Serum analysis was performed using high-sensitivity ELISA assay kits obtained from R&D systems. A.C. Serum from 3 mice at day 76 (CRC) (blue) and at day 91 (NSCLC) (red) was analyzed for hIL8 and hVEGF serum levels. Results are shown as bar graphs representing the mean cytokine concentration in pg/mL+/−SD from n=3 mice/group. B.D: After Kineret treatment was stopped in the CRC model the inventors compared cytokine levels from three individual mice classified as a high, a moderate and a low responder to Kineret on the basis of tumor progression rates Under all conditions, hVEGF levels remained stable, whereas hIL-8 levels declined dramatically in Kineret treated mice and were observed to directly correlate with tumor burden. These results will be confirmed with serum from a second in vivo experiment.

FIG. 14: Inhibition of IL1R1 by a neutralizing antibody reduces TAM supported tumor sphere formation in a dose dependent manner.

A. Spontaneous mammary tumors from HER/neu transgenic mice were isolated and tumor cells isolated. Tumor associated macrophages (TAMS) were sorted using CD11b and F4/80 together with the pan-hematopoietic marker CD45. A.B. HER2/neu cells were plated as tumor spheres alone and together with TAM. TAM alone served as a control. Mean and SD of tumor sphere number per well from triplicates is shown as a bar graph. TAM supported HER2/neu sphere growth in a TAM-cell number dependent fashion. C. An anti-murine IL1R1 hamster IgG Mab was added to HER2/neu tumor spheres at 0.5 μg/mL and 10 μg/mL (left panel) as well as to HER2/neu+ TAM co-cultures. Tumor sphere numbers are shown as bar graphs of mean and SD from triplicates. TAM-supported sphere growth is inhibited in a dose is dependent manner under anti-IL1R1 treatment.

### DETAILED DESCRIPTION OF THE INVENTION

If not otherwise pointed out, the terms and phrases used in this invention preferably have the meanings and definitions as given below. Moreover, these definitions and meanings describe the invention in more detail, preferred embodiments and/or aspects included.

“Cancer stem cells (CSC)”: A consensus panel convened by the American Association of Cancer Research has defined a CSC as “a cell within a tumor that possesses the capacity to self-renew and to cause the heterogeneous lineages of cancer cells that comprise the tumor.” It should be noted that this definition does not indicate the source of these cells—these tumor-forming cells could hypothetically originate from stem, progenitor, or differentiated cells. As such, the terms “tumor-initiating cell” or “cancer-initiating cell” are sometimes used instead of “cancer stem cell” to avoid confusion. Tumors originate from the transformation of normal cells through the accumulation of genetic modifications, but it has not been established unequivocally that stem cells are the origin of all CSCs. The CSC hypothesis therefore does not imply that cancer is always caused by stem cells or that the potential application of stem cells to treat conditions such as heart disease or diabetes, as discussed in other chapters of this report, will result in tumor formation. Rather, tumor-initiating cells possess stem-like characteristics to a degree sufficient to warrant the comparison with stem cells; the observed experimental and clinical behaviors of metastatic cancer cells are highly reminiscent of the classical properties of stem cells.

Thus, CSC according to this invention can be regarded as a subpopulation (1-10%, preferably 2-5%) of cells within a tumor tissue including solid tumors and metastases, that are functionally and optionally phenotypic different from standard tumor tissue cells. CSC are tumorigenic, that means they can generate new tumor cells. CSC show long-term self renewal ability and generate differentiated tumor bulk populations. CSC are also characterized by an enhanced ability to show resistance to chemotherapy and/or radiotherapy. Moreover, CSC can support metastasis by is driving the spread of disease to distinct organs. There are several hypothesis trying to explain the origin of CSC. CSC may arise from (i) stem cells, from (ii) progenitor cells, and (iii) from differentiated mature cells. CSC according to the invention can comprise other tumor cells, which are not CSC, in a range between 0-30%.

The term “tumor cell” or “cancer cell” relates, if not differently specified, to cells of uncontrolled growth, which are not cancer stem cells. Tumor or cancer cells may comprise CSC, in a range between 0-30%. Tumor cells represent the bulk population of ordinary tumor tissue.

The term “cancer” describes a group of diseases that are characterized by uncontrolled cellular growth, cellular invasion into adjacent tissues, and the potential to metastasize if not treated at a sufficiently early stage. These cellular aberrations arise from accumulated genetic modifications, either via changes in the underlying genetic sequence or from epigenetic alterations (e.g., modifications to gene activation- or DNA-related proteins that do not affect the genetic sequence itself). Cancers may form tumors in solid organs, such as the lung, brain, or liver, or be present as malignancies in tissues such as the blood or lymph. Tumors and other structures that result from aberrant cell growth, contain heterogeneous cell populations with diverse biological characteristics and potentials. In fact, cancerous tissues are sufficiently heterogeneous that the researcher will likely identify differences in the genetic profiles between several tissue samples from the same specimen. While some groupings of genes allow scientists to classify organ- or tissue-specific cancers into subcategories that may ultimately inform treatment and provide predictive information, the remarkable complexity of cancer biology continues to confound treatment efforts.

By means of the pharmaceutical compositions according of the present invention tumors can be treated such as tumors of the breast, heart, lung, small intestine, colon, spleen, kidney, bladder, head and neck, ovary, prostate, brain, pancreas, skin, bone, bone marrow, blood, thymus, uterus, testicles, cervix, and liver. More specifically the tumor is selected from the group consisting of adenoma, angio-sarcoma, astrocytoma, epithelial carcinoma, germinoma, glioblastoma, glioma, hamartoma, hemangioendothelioma, hemangiosarcoma, hematoma, hepato-blastoma, leukemia, lymphoma, medulloblastoma, melanoma, neuroblastoma, osteosarcoma, retinoblastoma, rhabdomyosarcoma, sarcoma and teratoma. More preferably, the tumor/cancer is selected from the group consisting of intracerebral cancer, head-and-neck cancer, rectal cancer, astrocytoma, preferably astrocytoma grade II, III or IV, glioblastoma, preferably glioblastoma multiforme (GBM), small cell lung cancer (SCLC) and non-small cell lung cancer (NSCLC), preferably non-small cell lung cancer (NSCLC), metastatic melanoma, metastatic androgen independent prostate cancer (AIPCa), metastatic androgen dependent prostate cancer (ADPCa), breast cancer and colorectal cancer (CRC).

A “receptor” or “receptor molecule” is preferably a soluble or membrane bound or membrane associated protein or glycoprotein comprising one or more domains to which a ligand binds to form a receptor-ligand complex. By binding the ligand, which may be an agonist or an antagonist the receptor is activated or inactivated and may initiate or block pathway signaling.

By “ligand” or “receptor ligand” is preferably meant a natural or synthetic compound which binds a receptor molecule to form a receptor-ligand complex. The term ligand includes agonists, antagonists, and compounds with partial agonist/antagonist activity.

An “agonist” or “receptor agonist” is preferably a natural or synthetic compound which binds the receptor to form a receptor-agonist complex by activating said receptor and receptor-agonist complex, respectively, initiating a pathway signaling and further biological processes.

By “antagonist” or “receptor antagonist” is preferably meant a natural or synthetic compound that has a biological effect opposite to that of an agonist. An antagonist binds the receptor and blocks the action of a receptor agonist by competing with the agonist for receptor. An antagonist is defined by its ability to block the actions of an agonist. A receptor antagonist may be also an antibody or an immunotherapeutically effective fragment thereof. Preferred antagonists according to the present invention are cited and discussed below.

The term “antibody” or “immunoglobulin” herein is preferably used in the broadest is sense and specifically covers intact monoclonal antibodies, polyclonal antibodies, multispecific antibodies (e.g. bispecific antibodies) formed from at least two intact antibodies, and antibody fragments (such as Fc, Fab,F(ab′)2, scFv, etc.) so long as they exhibit the desired biological activity. The term generally includes hetero-antibodies which are composed of two or more antibodies or fragments thereof of different binding specificity which are linked together. The term includes in addition antibody fusion proteins, which are composed of an antibody or antibody fragment and a polypeptide or protein recombinantly fused to the antibody or antibody fragment, and immunoconjugates, wherein the antibody or antibody fragment is chemically linked to a chemical entity The term includes further human, humanized and chimeric antibodies.

The term “cytotoxic agent” as used herein preferably refers to a substance that inhibits or prevents the function of cells and finally causes destruction of cells and cell death, especially tumor cell death. The term is preferably intended to include radioactive isotopes, chemotherapeutic agents, and toxins such as enzymatically active toxins of bacterial, fungal, plant or animal origin, or fragments thereof. The term may include also members of the cytokine family, preferably IFNγ as well as anti-neoplastic agents having also cytotoxic activity.

The term “cytostatic agent” as used herein preferably refers to a substance, including antibodies, antibody fragments, immunoconjugates or antibody fusion proteins, that inhibits or prevents the function of cells, or retards cellular activity and multiplication and finally causes prevention of cell growth without killing them.

The term “chemotherapeutic agent”, “chemotherapeutical agent” or “anti-neoplastic agent” is regarded according to the understanding of this invention preferably as a member of the class of “cytotoxic agents” or “cytostatic agents” as specified above, and includes chemical agents that exert anti-neoplastic effects, i.e., prevent the development, maturation, or spread of neoplastic cells, directly on the tumor cell, e.g., by cytostatic or cytotoxic effects, and not indirectly through mechanisms such as biological response modification. Suitable chemotherapeutic agents according to the invention are preferably natural or synthetic chemical compounds, but biological molecules, such as proteins, polypeptides etc. are not expressively excluded. There are large numbers of anti-neoplastic agents available in commercial use, in clinical evaluation and in pre-clinical development, which could be included in the present invention for treatment of tumors/neoplasia. Examples of chemotherapeutic or agents include alkylating agents, for example, nitrogen mustards, ethyleneimine compounds, alkyl sulphonates and other compounds with an alkylating action such as nitrosoureas, cisplatin and dacarbazine; antimetabolites, for example, folic acid, purine or pyrimidine antagonists; mitotic inhibitors, for example, vinca alkaloids and derivatives of podophyllotoxin; cytotoxic antibiotics and camptothecin derivatives. Preferred chemotherapeutic agents or chemotherapy include amifostine (ethyol), cisplatin, dacarbazine (DTIC), dactinomycin, mechlorethamine (nitrogen mustard), streptozocin, cyclophosphamide, carmustine (BCNU), lomustine (CCNU), doxorubicin (adriamycin), doxorubicin lipo (doxil), gemcitabine (gemzar),daunorubicin, daunorubicin lipo (daunoxome), procarbazine, mitomycin, cytarabine, etoposide, methotrexate, 5-fluorouracil (5-FU), vinblastine, vincristine, bleomycin, paclitaxel (taxol), docetaxel (taxotere), aldesleukin, asparaginase, busulfan, carboplatin, cladribine, camptothecin, CPT-11,10-hydroxy-7-ethyl-camptothecin (SN38), dacarbazine, floxuridine, fludarabine, hydroxyurea, ifosfamide, idarubicin, mesna, interferon alpha, interferon beta, irinotecan, mitoxantrone, topotecan, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil and combinations thereof. The preferred chemotherapeutic agents used in combination with any engineered antibody according to the invention may be e.g. methotrexate, vincristine, adriamycin, cisplatin, non-sugar containing chloroethylnitrosoureas, 5-fluorouracil, mitomycin C, bleomycin, doxorubicin, dacarbazine, taxol, fragyline, Meglamine GLA, valrubicin, carmustaine, UFT (Tegafur/Uracil), ZD 9331, Taxotere/Decetaxel, Fluorouracil (5-FU), vinblastine, and other well compounds from this class.

The term “therapeutically effective” or “therapeutically effective amount” preferably refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can, for example, be measured by assessing the time to disease progression (TTP) and/or determining the response rate (RR).

The therapeutic effective amount of antibodies used in this invention, including the anti-IL1IR1 polypeptides, is for an adult of about 70 kilograms in the range between about 50 to 4000 milligrams per dose, with a preferred range of about 100 to 1000 milligrams per dose. The most preferred dose is about 200-500 milligrams for a 70 kg adult treated once or twice per month.

The therapeutic effective amount of a chemotherapeutic agents as mentioned herein are is as a rule a dose between 10 mg/kg and 100 mg/kg.

Administration is preferably once per two weeks or once per month, but may be more or less frequent depending on the pharmacokinetic behavior of the respective agent in a given individual.

The term “radiation therapy” and related terms mean according to this invention the administration or delivery of focal ionizing radiation, wherein 20 to 50 Gray (Gy), preferably 25 to 40 Gy, more preferably 28 to 25 Gy, for example about 28 Gy, about 30 Gy or about 35 Gy are administered or delivered to the patient, preferably in fractions of 0.5 to 5 Gy, more preferably 0.8 to 3 Gy and especially 1 to 2.5 Gy, for example about 1.0, about 1.3 Gy, about 1.6 Gy, about 1.8 Gy, about 2.0 Gy, about 2.5 Gy or about 3.0 Gy, per per administration or delivery, which is preferably also the amount of radiation per day on which the administration or delivery of the radiation takes place. Accordingly, an administration or delivery of 1.5 to 2.5 Gy and preferably 1.8 to 2.2 Gy per day for 2 or 3 days within one week is preferred. Accordingly, an administration or delivery of 0.7 to 1.3 Gy and preferably 0.9 to 1.2 Gy per day for 3 to 6 days, preferably for 5 days and more preferably 5 consequtive days, within one week, is also preferred. Generally, the administration or delivery of 1.0 to 3.0 Gy, preferably about 1.0, about 2.0 Gy or about 3.0 Gy per day for 2 or 3 days within one week is especially preferred. The kind of application of focal radiotherapy as described above is preferred in the treatment of cancer types selected from the group consisting of small cell lung cancer and non-small cell lung cancer, preferably non-small cell lung cancer, breast cancer, metastatic melanoma, prostate cancer, and colorectal cancer.

A “pharmaceutical composition” of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediamine-tetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

“IL-1R inhibitors”: Suitable IL-1R inhibitors according to the invention are polypeptides, preferably human, chimeric or humanized monoclonal antibodies, as well as IL-1R receptor antagonist (IL-1RA), which is a natural inhibitor of IL-1R1. Suitable antibodies are anti-IL-1R1 antibodies or anti-IL-1beta antibodies or antibodies directed against IL-1R accessory protein (IL-1RaP) or both IL-1RaP and IL-1R1, bispecific antibodies included. Suitable inhibitors of IL-1R according to the invention include further fusion molecules of above-specified antibodies with other targeting or functionally effective molecules, as well as fusion molecules functioning as a trap for the natural IL-1R1 ligand IL-1beta, thus preventing binding of IL-1beta to the IL-1 receptor.

Examples of such polypeptides according to the invention are known in the art. WO 2004/039951 and WO 2000/018932 describe an IL-1beta trap consisting of IL-1R1 fused to IL-1RaP (Acralyst). WO 1989/011540 and WO 2001/042305 describe recombinant natural and modified IL-1RA (anakinra/Kineret). WO 2004/022718 disclose anti-IL-1R antibodies (AMG-108). A further anti-IL-1R antibody is described in WO 2005 023872 (2D8). WO 2002/016436 describe a human anti-IL-1beta antibody (canakinumab/Ilaris), and WO 2007/002261 disclose another humanized antibody directed against IL-1beta. In WO 2010/052505 further anti-IL-1R1 antibodies are disclosed.

All of these polypeptides/antibodies described above were and are used in practice for the treatment of inflammatory diseases only.

The data provided by this invention and demonstrated in detail below by the inventors show that signalling along the IL-1beta/IL-1R1 axis plays an important role in CSC, tumor maintenance and metastasis. Therefore, inhibition of IL-1R1 on CSC is a valid and promising target for the treatment of primary and metastatic disease in oncology.

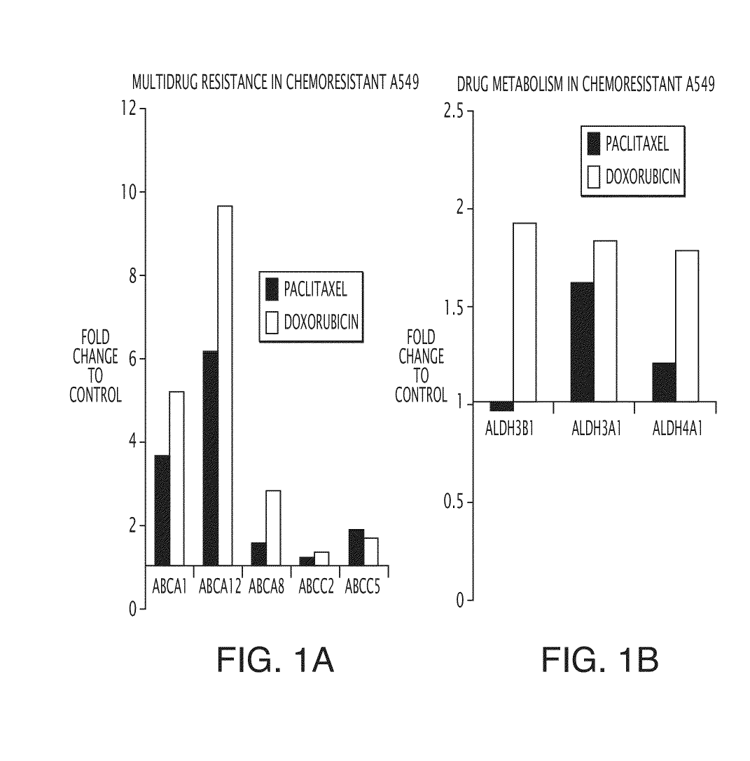

Assuming that high dose chemotherapy will eradicate the proliferative bulk of tumor cells and only leave cells with inherent resistance, the inventors treated the human A549 non small cell lung cancer (NSCLC) cell line with high dose Doxorubicin and Paclitaxel, recovered the surviving cells by density gradient centrifugation and prepared RNA extracts for further gene expression analysis on the Affymetrix microarray platform. The samples were analyzed by gene expression microarray huU 133 2.0plus from Affymetrix and then further analyzed for differential expression. Untreated/wild-type (WT) cells were compared with Paclitaxel and Doxorubicin enriched A549. As shown below stem cell markers as ABC-transporters (FIG. 1A) and ALDH-isoforms for rapid efflux and metabolization (FIG. 1B) are up-regulated in high dose chemotherapy-selected tumor cells.

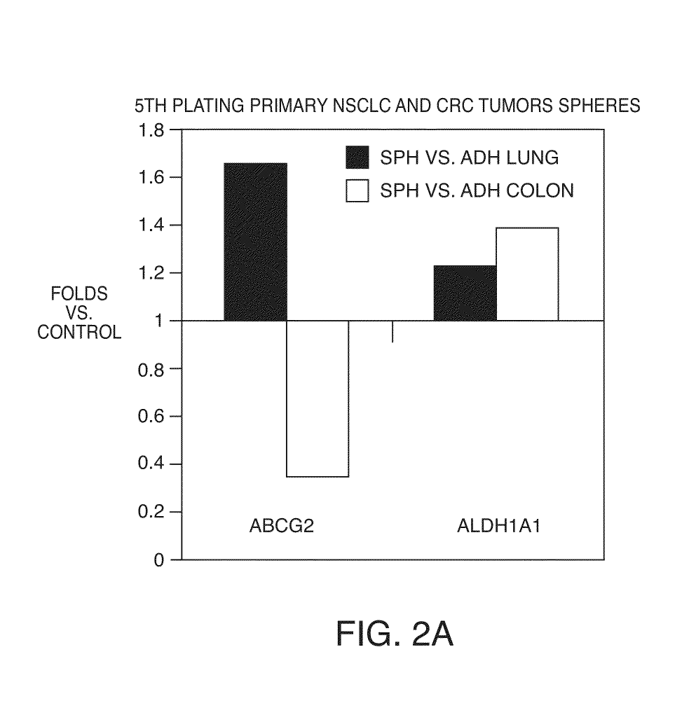

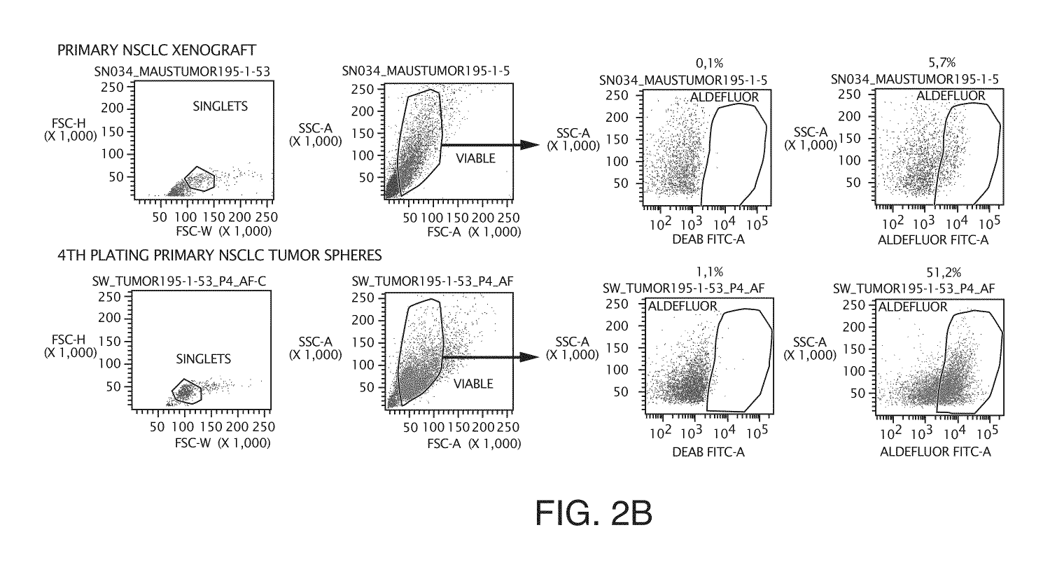

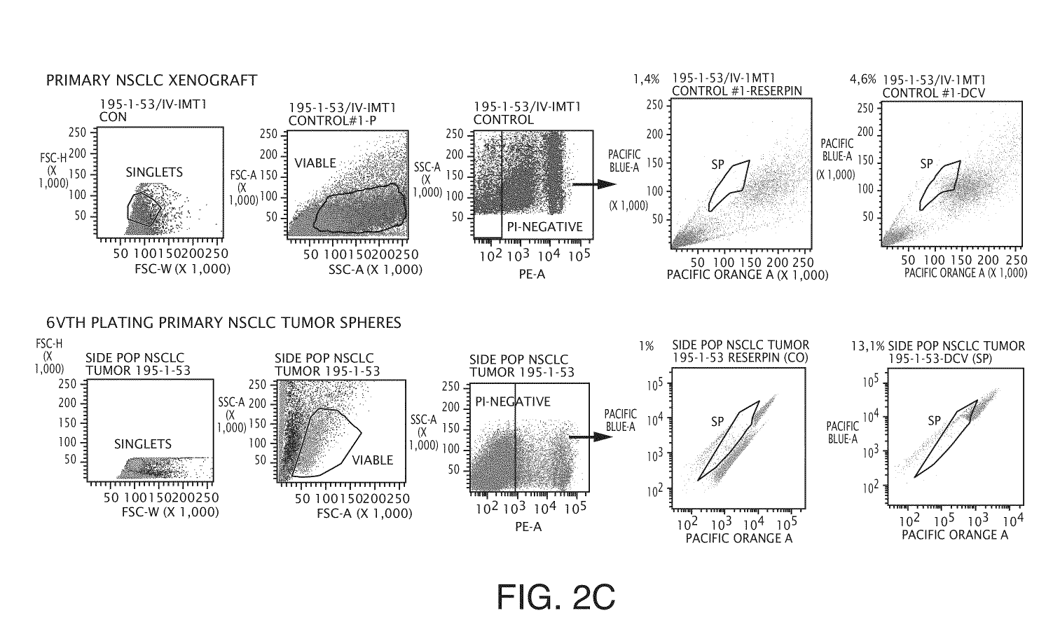

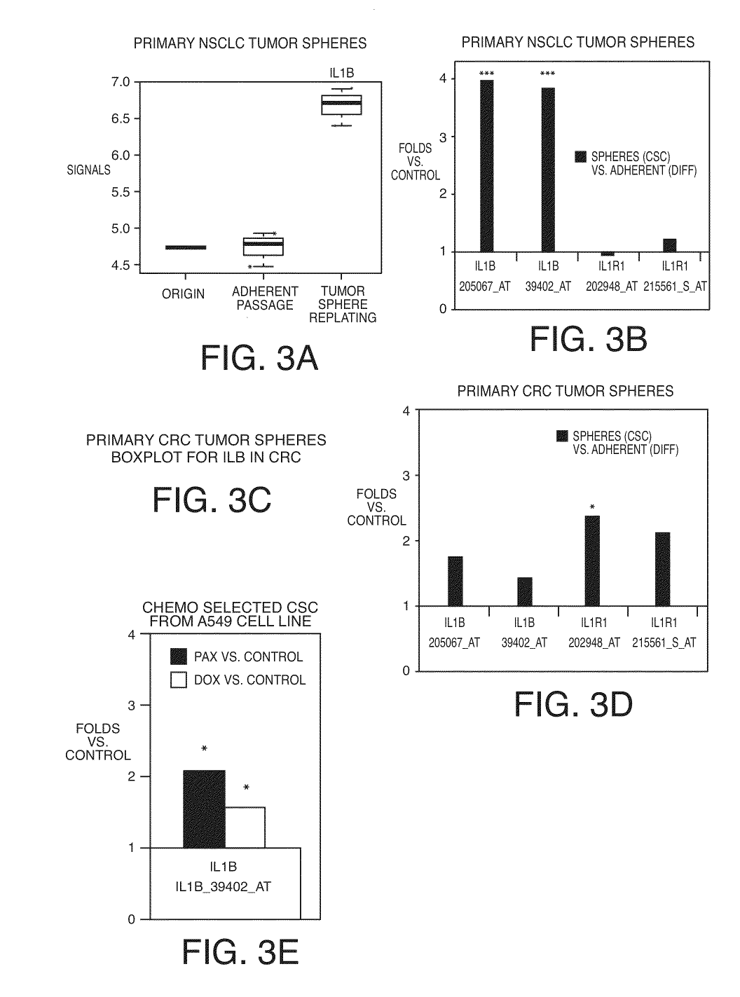

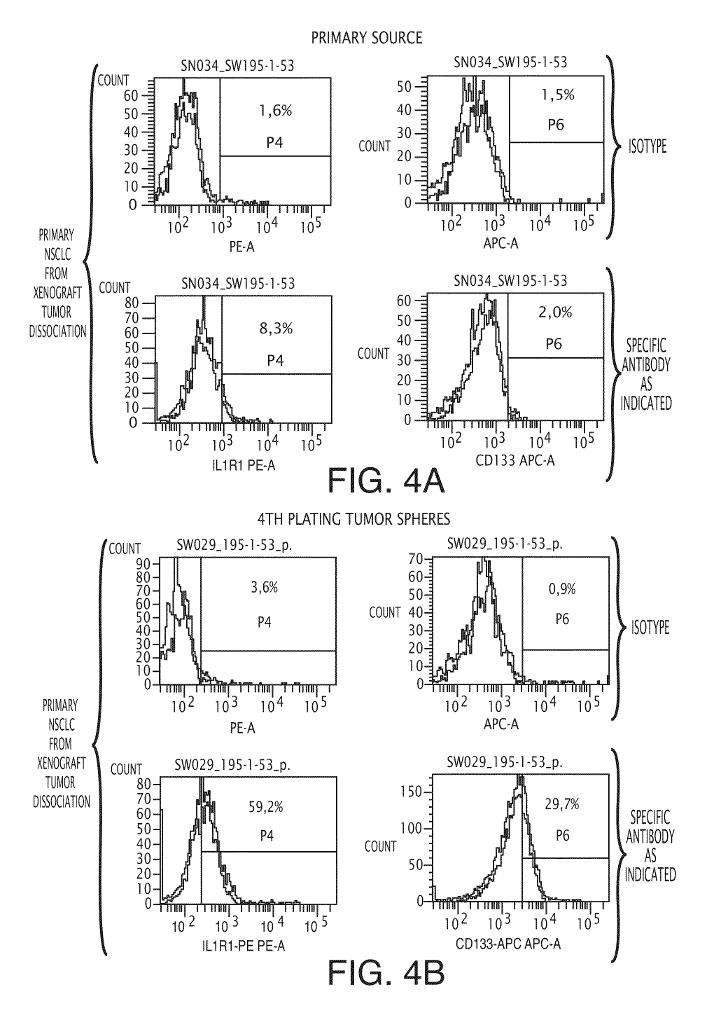

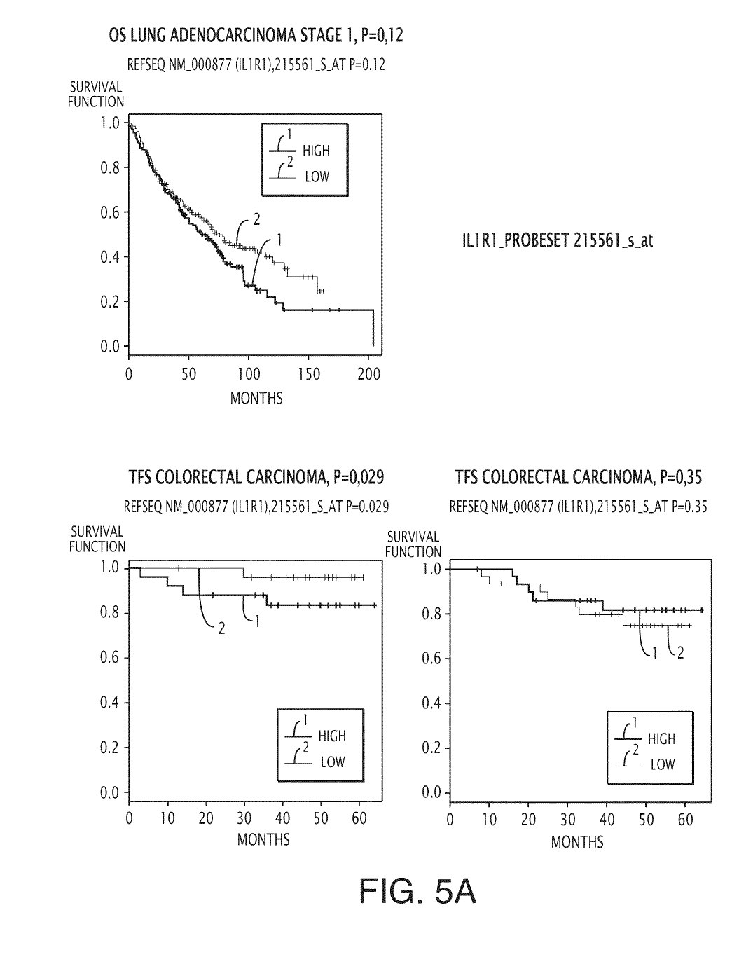

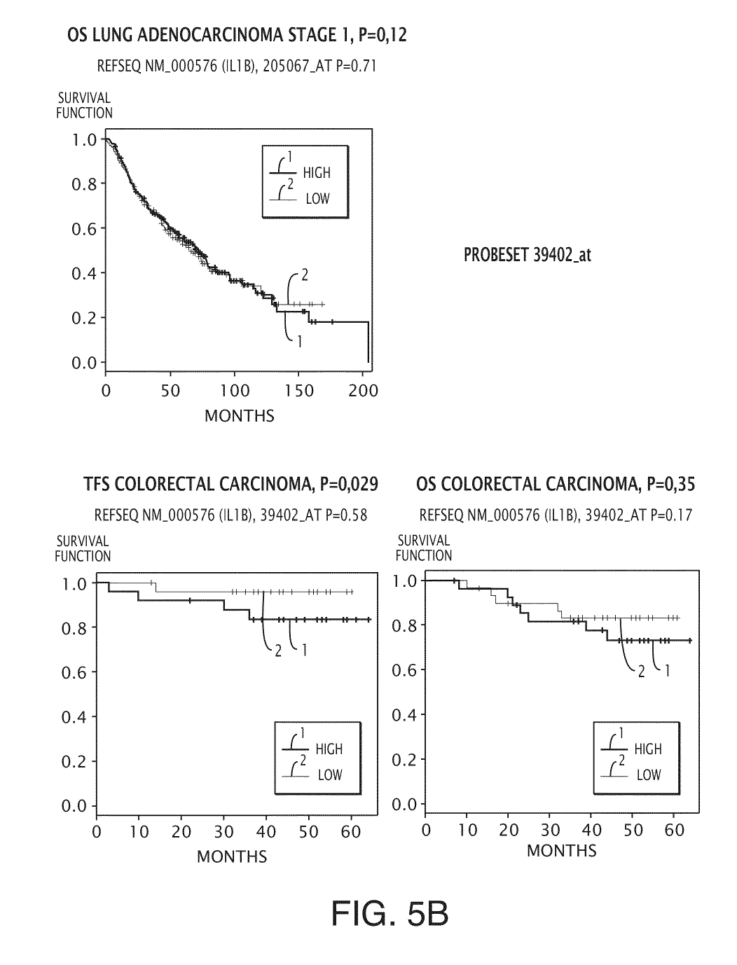

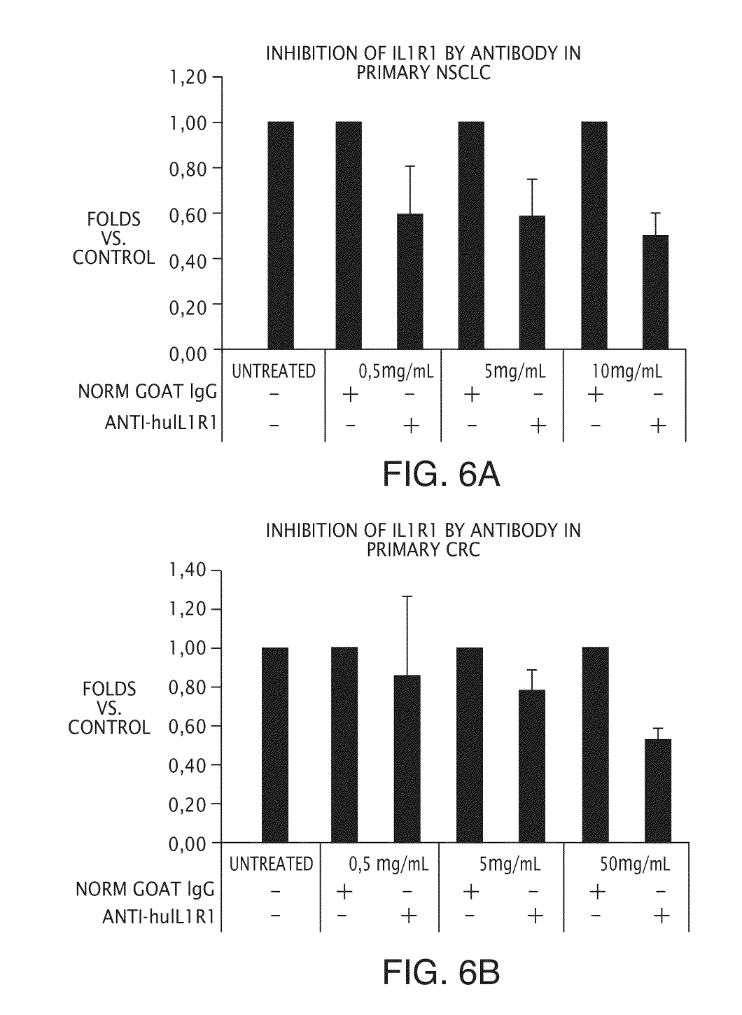

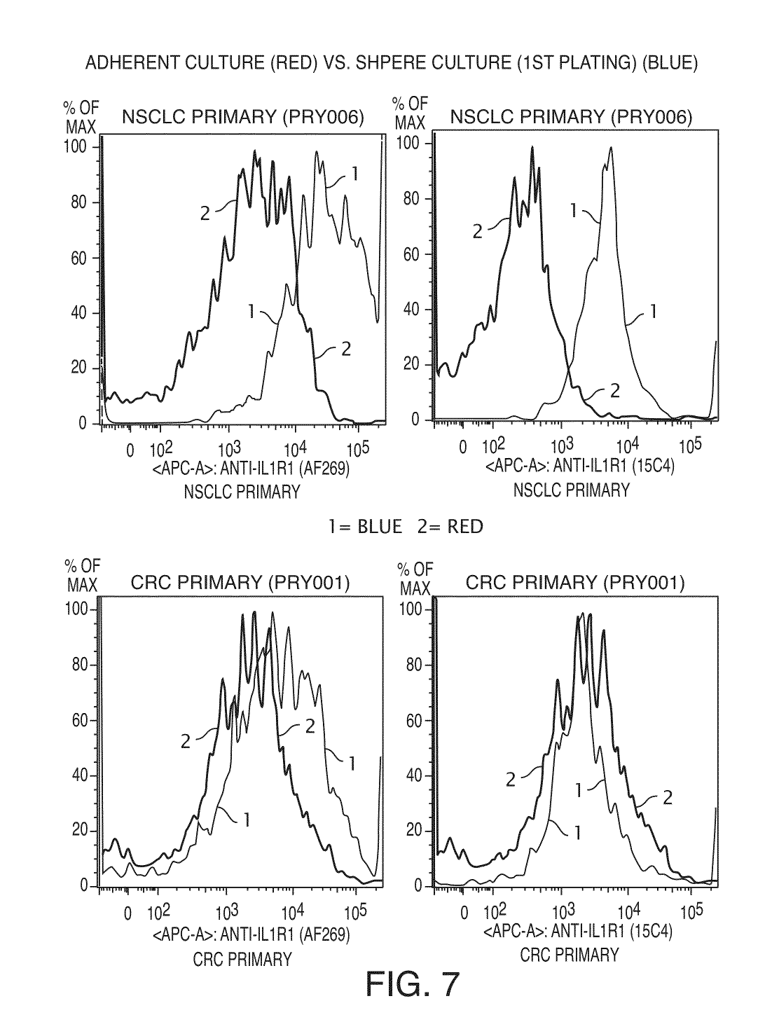

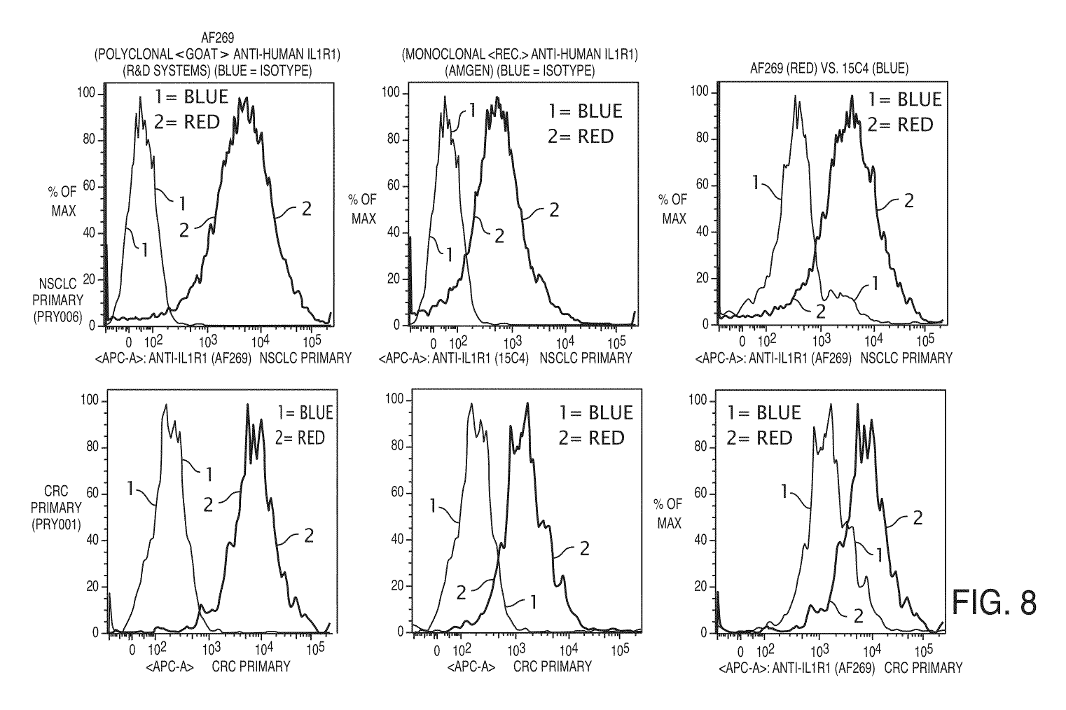

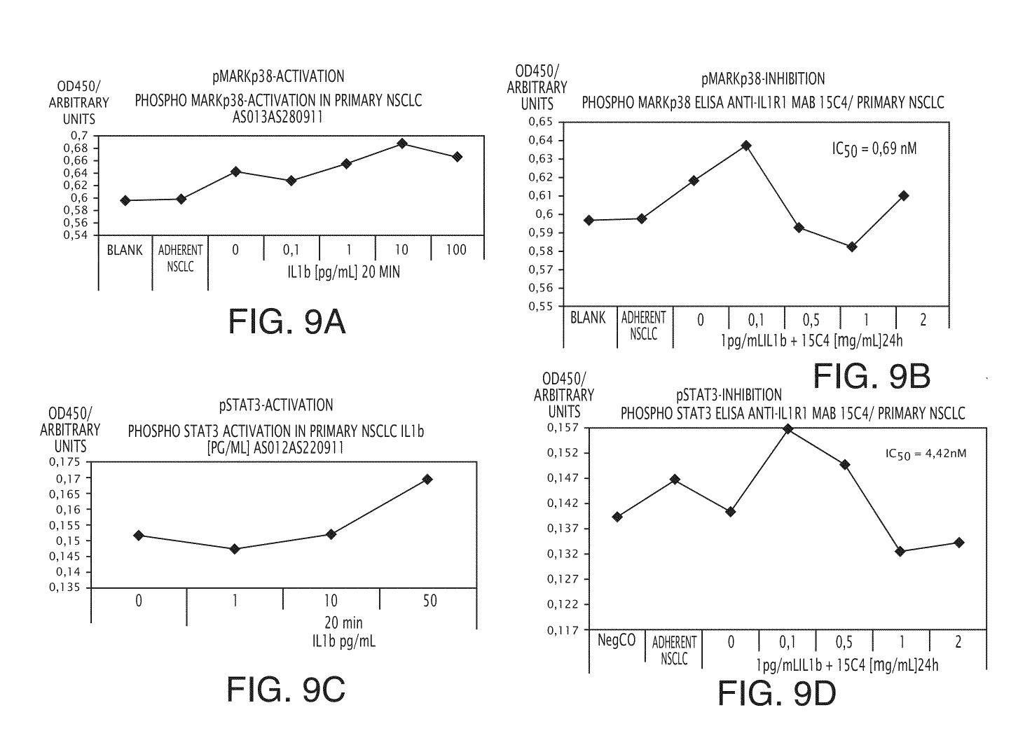

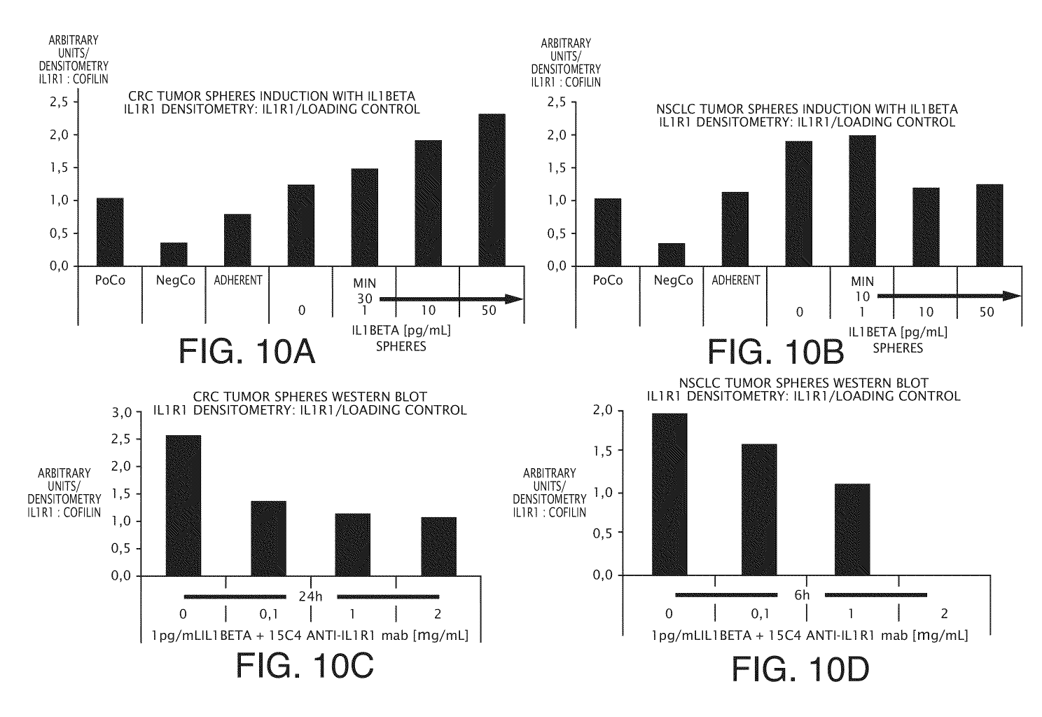

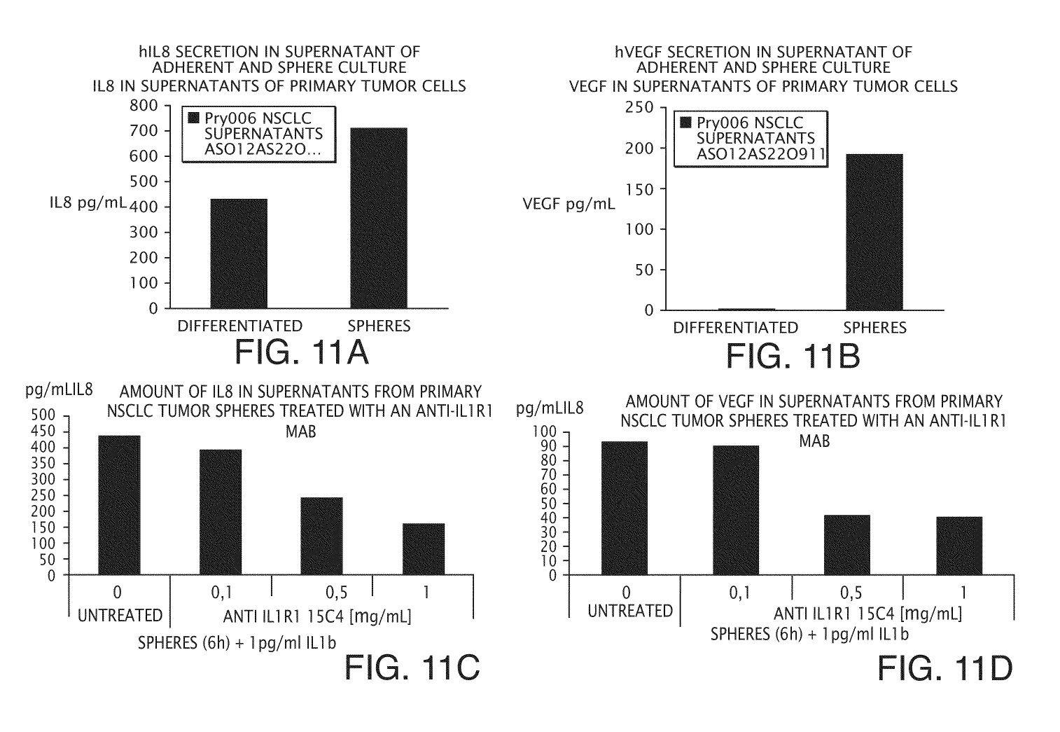

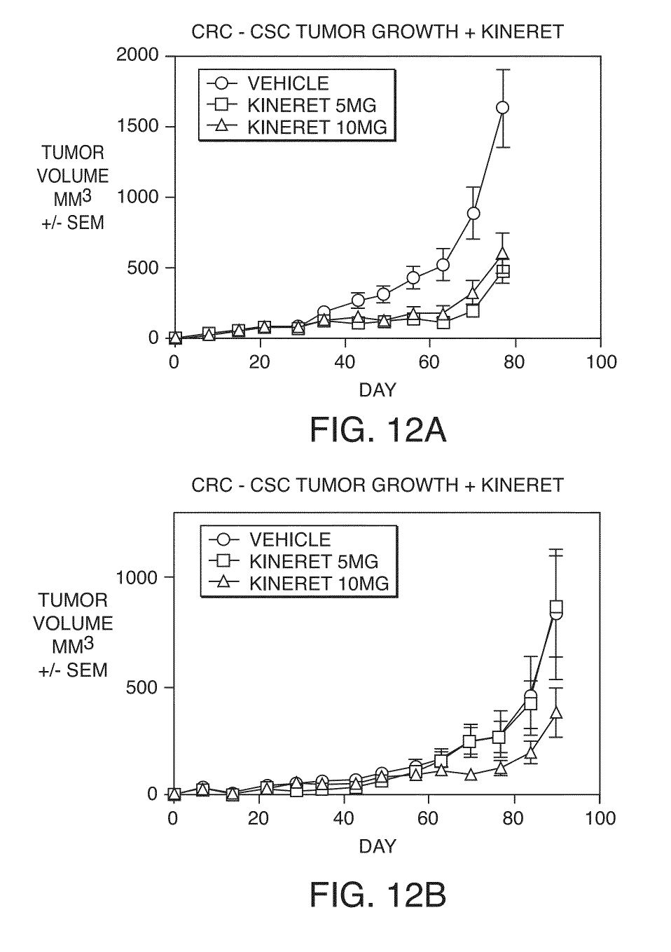

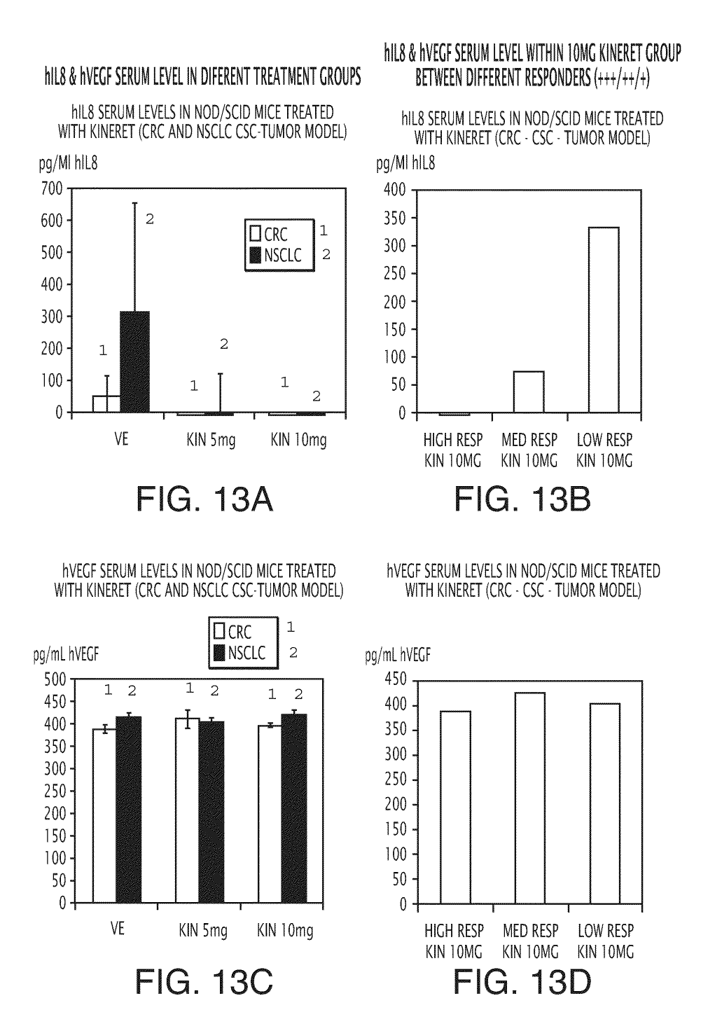

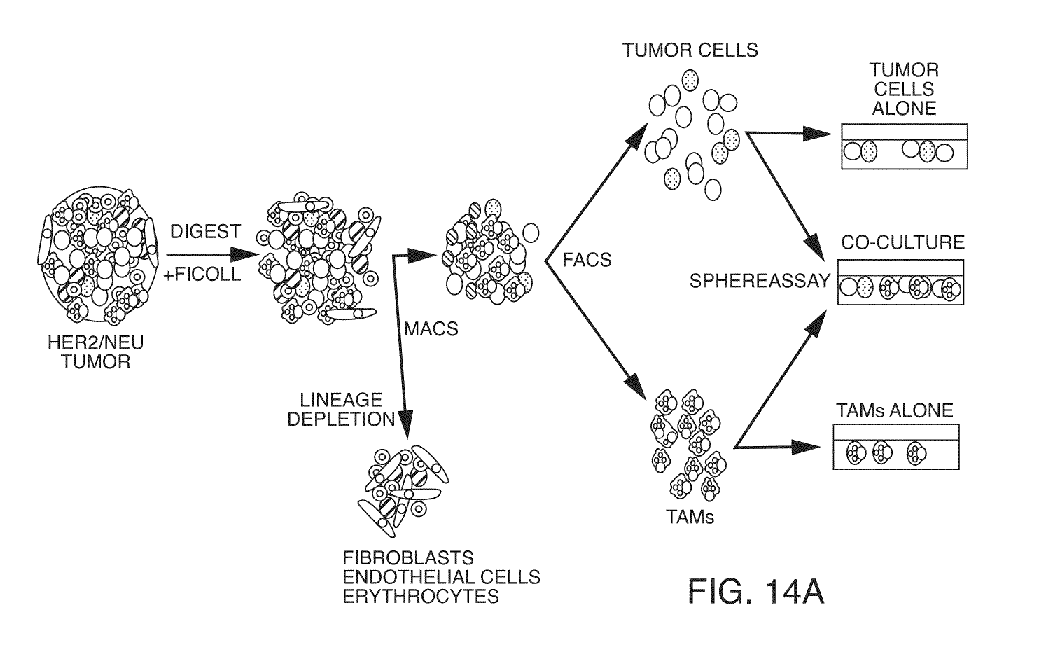

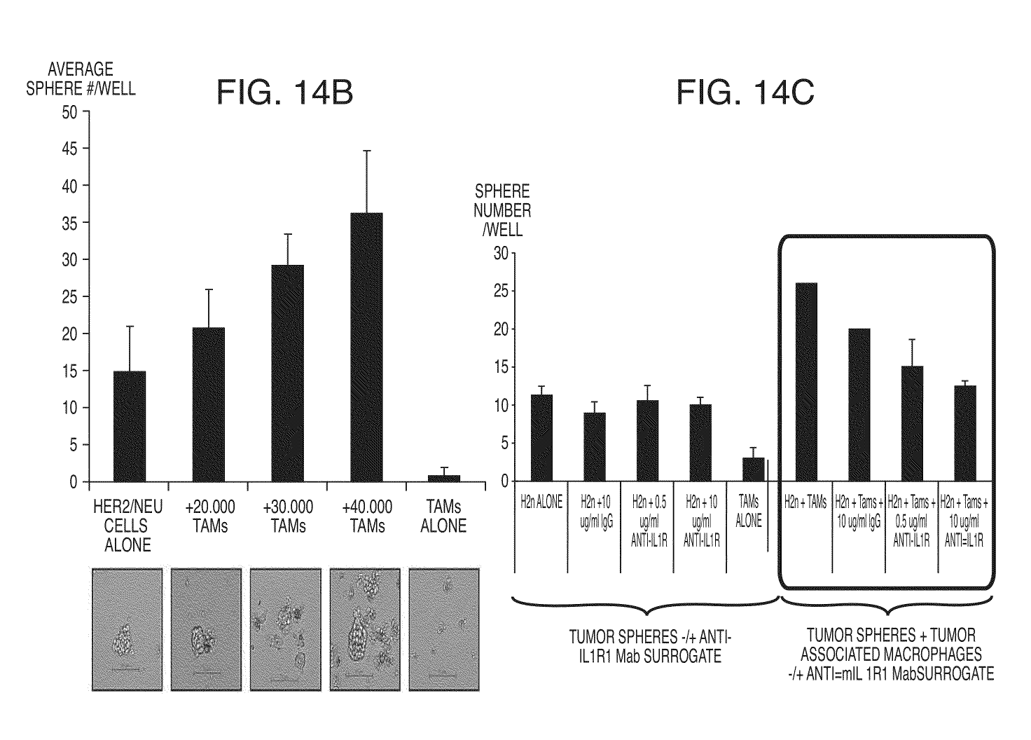

In a second step the inventors built a model system to analyse CSC versus the differentiated tumor bulk cells. The tumor sphere assay was used to enrich for CSC by serial re-plating, whereas the adherent condition with serum was taken to contain is higher amounts of differentiated cells. As shown in FIG. 2A stem cell markers ABCG2 and ALDH1A1 were up-regulated in the tumor sphere assay compared to adherent conditions. These results could be proven in the two golden standard functional assays for stem cells: the Aldefluor (AF)— Assay from Stem Cell Technologies (SCT) and the side population (SP) assay [Goodell, M. A., et al., J Exp Med, 1996. 183(4): p. 1797-806]. Both CSC-markers are up-regulated when cells are plated as tumor spheres, which shows that the tumor sphere assay is a bona fide stem cells assay to functionally enrich for CSC (FIGS. 2B and C).

RNA was extracted from the initial cells before plating (P0) and then from consecutive re-plating rounds of tumor spheres as well as from consecutive adherent passages. Primary patient material form either colorectal carcinoma (CRC) or NSCLC were cultured this way.

The samples were also analyzed by gene expression microarray huU133 2.0plus from Affymetrix and then further analyzed for differential expression. Here the adherent passages were compared with the tumor sphere platings and also linearity of identified differentially expressed genes were considered. Linearity means consistent up or down regulation in tumor spheres compared to adherent passages.

The inventors identified various differentially expressed genes from which the inventors selected the ones which are secreted or located to the surface of the cells. IL-1beta was one of the most stringent regulated genes in primary NSCLC (FIGS. 3A and B) and was also up-regulated in CRC (FIGS. 3C and D). Expression of IL-1beta is low and stable in adherent/differentiated passages, whereas in tumor spheres it is significantly up-regulated when compared to adherent/differentiated. Therefore it is clear that under differentiating conditions IL-1beta is not playing a major role whereas in tumor spheres which represent CSC conditions IL-1beta is up-regulated assuming that it is of major importance. It was also found that the receptor to for IL-1, the IL-1 receptor 1 (IL-1R1) is slightly up-regulated in NSCLC spheres (FIG. 3B). In primary CRC IL1beta was only slightly regulated whereas the IL-1R1 was significantly up-regulated (FIG. 3D). The cytokine IL-1beta was also up-regulated in chemotherapy-selected cells, which provided further validation of the functional selection methods to enrich for CSC (FIG. 3E).

The mRNA levels of IL-1beta and its respective receptor were up-regulated in functionally enriched CSC. To further evaluate the relevance on a protein level the inventors analyzed the primary patient material derived NSCLC samples. Patient material was in vivo propagated as subcutaneous (s.c.) xenografts in immune-compromised mice. Tumors were dissociated and further analyzed as well as re-plated in a tumor sphere assay. Protein expression of IL-1R1 was present on parental patient tumor cells (FIG. 4A) and derived tumor spheres. Also IL-1R1 protein expression on the surface is up-regulated upon tumor sphere re-plating which is consistent with our findings on the RNA/microarray-level. The CSC-marker CD133 is also up-regulated in the same re-plated tumor spheres, what indicates that IL-1R1 is a CSC-associated surface molecule (FIG. 4B).

Assuming, IL-1beta and its respective receptor play a major role in tumor maintenance and metastasis the inventors used an in silico approach to evaluate the effects of IL-1beta and IL-1R1 on disease free and overall survival. For the probe sets 215561_s_at for IL-1beta and 39402_at for IL-1R1 the inventors found that low expression of either IL-1beta correlated with a survival benefit in lung adenocarcinoma stage I patients and with a prolonged disease free survival in CRC.

Overall survival in CRC showed no significant differences with high or low expression of IL-1B (FIG. 5A). High expression of IL-1R1 showed a slight survival benefit in survival in lung adenocarcinoma stage I and a clear benefit in disease free and overall survival in CRC (FIG. 5B).

IL-1R1RNA and protein expression on the surface is up-regulated upon tumor sphere re-plating whereas the RNA expression under adherent conditions is stable. The tumor sphere assay was established and has been shown to enrich for CSC and CSC-like progenitor cells whereas differentiated cells die. Therefore it can be assumed that the IL-1beta/IL-1R1 signalling might play an important role in the to development or maintenance of the CSC phenotype. To test whether IL-1beta/IL-1R1 signalling is important in tumor spheres, the inventors plated either primary NSCLC or CRC in a tumor sphere assay and treated the cells with a dose range of 0.5, 5 and either 10/50 μg/mL normal goat IgG1 or the neutralizing goat-anti-human-IL-1R1 antibody. Here the inventors show that inhibition of IL-1R1 by a neutralizing antibody reduces the sphere formation ability of the plated cells in a dose dependent manner in NSCLC (FIG. 6A) and CRC patient derived cells (FIG. 6B).

FIGS. 7 and 8 depict the upregulated expression of IL-1R1 on CRC and NSCLC tumors and its detection by different antibodies.

The inhibitory effect of the anti-IL-1R1 antibody on IL-1beta was also shown (FIG. 9, 10, 14).

### EXAMPLES

### Example 1

### Identification of IL1/IL1R1 Signaling as a CSC-Relevant Pathway

CSC are characterized by their high expression of drug-efflux transporters and detoxifying enzymes. The inventors therefore hypothesized that they could select for cells with innate CSC-like properties by treating cancer cell lines with supraphysiological, high dose chemotherapy. This approach eradicates the hyperproliferative bulk and leaves cells with inherent (not acquired) resistance. The inventors treated the A549 non small cell lung cancer (NSCLC) cell line with high dose Doxorubicin and Paclitaxel, recovered the surviving cells by density gradient centrifugation and prepared RNA extracts for further gene expression analysis on the Affymetrix microarray platform. The samples were analyzed by gene expression microarray huU133 2.0plus from Affymetrix and then further analyzed for differential expression by comparison against untreated/wildtype (WT) cells. As shown below, expression of ABC-transporters (drug efflux pumps) (FIG. 1A) and ALDH-isoforms (detoxifying enzymes) (FIG. 1B) were upregulated in high dose chemo-selected cells. The approach according to the invention thus successfully enriched for a cell population expressing high levels of known CSC markers.

Another characteristic of CSC is their ability to grow as spheroid structures when placed in non-adherent culture conditions. The tumor-sphere assay promotes CSC self-renewal in vitro and prevents differentiation. Serial re-plating of tumor-spheres thus further enriches for CSC. By comparison, under adherent conditions, the bulk of cultured cells represent differentiated tumor cells. Using cancer cells derived from human primary NSCLC tumor material, the inventors performed a differential is analysis of gene expression patterns in serially re-plated tumor-spheres versus adherent cultures. As shown in FIG. 2 A stem cell markers ABCG2 and ALDH1A1 were upregulated in the tumor-spheres as compared to differentiated adherent cells. The CSC-properties of sphere-propagated cells were further proven using two ‘gold standard’ functional assays for stem cells: the Aldefluor (AF)— Assay from Stem Cell Technologies (SCT) and the side population (SP) assay [Godell et al., 1996, J. Exp. Med. 183(4): 1797)]. The AF+ and SP+ fractions were both substantially increased in tumor-spheres, confirming that the tumor sphere assay is a bona fide assay for functional enrichment of CSC (FIGS. 2 B and 2 C).

Using human primary CRC and NSCLC tumor material, the inventors made a differential analysis of gene expression that compared the initial cells before plating (P0), cells serially passaged as tumor-spheres, and cells serially passaged as adherent cultures. The samples were analyzed by gene expression microarray huU133 2.0plus from Affymetrix. The analysis considered the linearity of identified differentially expressed genes, with linearity being defined as a consistent up or down regulation in tumor spheres versus adherent passages.

Various differentially expressed genes were identified from which the ones were selected which encode proteins that are either secreted or located on the cell surface, as it was considered by the inventors that such proteins represent the most readily druggable targets. Expression of IL-1β was low and stable in adherent/differentiated passages and was identified as one of the most highly upregulated genes in primary NSCLC (FIGS. 3A and B) and was also mildly upregulated in CRC (FIGS. 3C and D). This suggested to us that IL-1β may play an important role in the biology of CSC. In support of this hypothesis we found that the IL1 receptor 1 (URI) was slightly upregulated in NSCLC spheres (FIG. 3B). In primary CRC, IL-1β was only slightly upregulated, however, the IL1R1 was significantly upregulated (FIG. 3D). IL-1β was also found to be upregulated in cells enriched for CSC-like characteristics by selection with high-dose chemotherapy (FIG. 3E).

### Example 2

### Expression of the IL-1β and IL Genes is Associated with Decreased Tumor-Free and Overall Survival in Cancer Patients

Assuming that IL-1β or its respective receptor play a major role in tumor maintenance and metastasis the inventors used an in silico approach to evaluate the effects of IL-1β and IL1R1 on disease free and overall survival. Using the probesets 215561_s_at for IL1R1 and 205067_at or 39402 at for IL1β it was found that low expression of IL1R1 correlated with a survival benefit in lung adenocarcinoma stage I patients and with a prolonged disease free survival in CRC. Overall survival in CRC was unchanged by differences in IL1R1 expression levels (FIG. 5A). High expression of IL1β showed a trend towards a survival benefit in lung adenocarcinoma stage I and a clear benefit in disease free and overall survival in CRC (FIG. 5B). The CRC and NSCLC indications were chosen for analysis based on the fact that our initial identification of IL-1β and IL1R1 expression in CSC was made using primary material derived from patients with these tumor types.

### Example 3

### IL1R1 is Expressed on Primary CRC and NSCLC Tumor Cells and is Upregulated Following Enrichment of CSC by Serial Tumor-Sphere Propagation

The RNA levels of IL-1B and its respective receptor were upregulated in functionally enriched CSC or CSC-like cells. To determine the expression of IL1R1 on a protein level a FACS analysis of patient-derived NSCLC primary tumor samples was performed. Prior to the analysis, the patient material was in vivo propagated as subcutaneous (s.c.) xenografts in immunocompromised mice. The xenograft tumors were enzymatically dissociated into single-cells and IL1R1 surface expression was detected using commercially available fluorophore-conjugated antibodies. IL1R1 expression in cells taken directly from the xenograft tumors was compared to xenograft-derived cells that were serially passaged as tumor-spheres. Protein expression of IL1R1 was present on both the parental xenograft tumor cells (FIG. 4A) and the xenograft-derived tumor spheres. However, IL1R1 protein expression was higher in the tumor spheres as compared to the parental cells; this is consistent with the findings on the mRNA expression level and suggests that CSC have a particular reliance on the IL1-pathway. The CSC-marker CD133 was also upregulated in the tumor spheres, providing further evidence that IL1R1 is a CSC-associated surface molecule (FIG. 4B).

### Example 4

### IL1R1 is Expressed on Human Primary Tumor-Derived CRC and NSCLC Cell Lines and its Expression is Upregulated in CSC-Enriching Tumor-Spheres

To further evaluate IL1R1 expression on the surface of primary NSCLC and CRC cells a commercially available polyclonal antibody (AF269; R&D systems) and a monoclonal antibody (15C4) derived from the Amgen patent WO2004022718A2 was used. The latter antibody was produced as a monoclonal hIgG4. Both antibodies were labelled with APC and expression was detected by flow cytometry. As can be seen in FIG. 7, both antibodies were able to detect an upregulation of IL1R1 expression in NSCLC cells after one round of sphere plating as compared with cells grown under adherent cell culture conditions (FIG. 7 upper panel), whereas, in the CRC cell line, the upregulation of IL1R1 in spheres was weaker and could only be detected with the polyclonal, but not the monoclonal antibody (FIG. 7 lower panel).

These results are consistent with the results described in FIG. 4 wherein IL1R1 expression was found to be upregulated in serially passaged xenograft-derived tumor-spheres as compared to freshly dissociated xenograft tumor cells.

FIG. 8 show the respective isotype controls for the NSCLC and CRC primary tumor-derived lines corresponding to the histograms shown in FIG. 10 (left and middle panel up and down). When comparing both anti-IL1R1 antibodies it is clearly observed that the polyclonal antibody has a greater sensitivity for target recognition most probably due to recognition of multiple epitopes (FIG. 7, right panel up and down).

### Example 5

### Antibody Blockade of L1R1 Inhibits Tumor-Sphere Formation In Vitro

It was observed according to the invention that CSC-enriched tumor spheres generated from patient-derived primary tumors are characterized by upregulated IL-1B and IL1R1 gene expression and IL1R1 protein expression. The inventors therefore came to the hypothesis that the IL-1 pathway plays an important role in the development and/or maintenance of the CSC phenotype. To test whether IL-1-signaling promotes tumor-sphere formation in vitro, the primary NSCLC and CRC cell lines were plated under tumor-sphere culture conditions and treated the cells across a range of concentrations with a neutralizing goat-anti-human-IL1R1 antibody or an isotype-matched negative control IgG. Here, it was shown that inhibition of IL1R1 by a neutralizing antibody reduced the sphere formation ability of the plated cells in a dose dependent manner in both the NSCLC (FIG. 6A) and CRC patient-derived cell lines (FIG. 6B). These results support the use of therapies directed against the IL-1 pathway as a means of inhibiting CSC expansion.

### Example 6

### IL1R1 Blockade Inhibits IL-1β-Stimulated MAPKp38 and STAT3 Phosphorylation

It was shown that antibody blockade of IL1R1 partially repressed the formation of tumor-spheres in cell lines derived from human primary tumors. The inventors therefore consider inhibition of IL-1 signalling to be a viable strategy for therapeutic intervention in cancer with a unique potential to inhibit the function of CSC. To evaluate potential biomarkers that could be used to inform pharmacological assessments of IL-1 pathway inhibitors, the status of mitogen activated protein kinase (MAPK) p38 and signal transducer and activator of transcription (STAT)₃ was analysed, which are known to be phosphorylated in the IL1/IL1R1 cascade. Detection of pMAPKp38 and pSTAT3 was performed using a Pathscan ELISA (Cell Signaling). Tumor-spheres were plated overnight in culture medium lacking growth factors and then added recombinant IL-1β across a dose-range followed by a 20 min incubation. As can be seen in FIGS. 9A and C both MAPKp38 and STAT3 are phosphorylated in a dose-dependent manner as an early event upon IL1 stimulation. In the absence of stimulation with IL-1β, pMAPKp38 is already detectable in tumor-sphere cultures, whereas it is not detected in adherently grown cells, indicating that a potential autocrine activation loop may be at work in CSC (FIGS. 9 A and B). Under the same assay conditions, treatment with the anti-IL1R1 15C4 antibody was observed to reduce the phosphorylation of both pMAPKp38 and pSTAT3 in a dose-dependent manner (FIGS. 9 B and D) with IC50s of 0.69 nM and 4.42 nM for pMAPKp38 and pSTAT3, respectively.

It can be concluded that the phosphorylation status of both proteins can be used as a read-out in a screening cascade directed at identifying compounds that inhibit the IL-1 pathway and also represent proximal biomarkers for in vitro and in vivo pharmacology studies. As the results in FIG. 9 are of preliminary nature, confirmation of the results needs to be obtained in order to build a stable screening cascade.

### Example 7

### Recombinant IL-1β Induces the Expression of IL in Tumor-Spheres, Whereas Antibody Blockade of IL Downregulates IL Expression

In regard to establishing a proximal biomarker the same samples described above (FIG. 9) were analyzed for IL1R1 expression levels by Western Blot. As can be seen in FIGS. 10 A and C expression of IL1R1 was present in tumor-spheres and was further increased upon stimulation with recombinant IL-1β. In CRC-derived tumor-spheres induction of IL1R1 followed a clearly dose-dependent trend, whereas in NSCLC spheres we observed a reduction of IL1R1 expression at concentrations above 1 pg/mL; this may indicate a negative feedback loop as a result of high intrinsic IL1β production in the primary NSCLC cells. This is consistent with our findings in the microarray analyses that demonstrated primary NSCLC spheres have significantly higher levels of IL-1β mRNA. IL-1β stimulated IL1R1 surface expression was reduced following treatment with the 15C4 anti-IL1R1 antibody, indicating that antibody engagement triggers receptor degradation (FIGS. 10 B and D).

### Example 8

### CSC-Enriched Tumor-Spheres Secrete the IL1-Responsive Cytokines hIL8 and hVEGF and Production of these Cytokines can be Inhibited by IL1R1 Blockade

It was already shown above that IL-1 biological activity can be detected in cell lines by monitoring the phosphorylation of MAP38K and STAT3. However, to support clinical translation it will be important to identify biomarkers that can be detected in easily collected liquid biomaterials such as urine or blood. Cytokines can be easily measured in serum using the ELISA method. IL-1β itself is a cytokine that can readily be monitored in serum samples. It is well established that IL-1 stimulates the secretion of additional cytokines like IL8 and VEGFα.

To test whether these cytokines can be used to monitor a response to anti-IL1R1 antibodies IL8 and VEGF levels were measured in supernatants (SN) from CSC-enriched tumor-sphere cultures. When the supernatants of differentiated (adherent) and tumor-sphere cultures were compared, a ˜1.7 fold induction of IL8 (FIG. 11A) was found and a striking induction of VEGF in SN from tumor-spheres (FIG. 11B). Based on this observation, IL8 and VEGF levels in SN from anti-IL1R1-Mab treated tumor spheres (FIGS. 11C and D) were measured. As can be seen below, blockade of IL1R1 reduces IL8-levels in a dose-dependent manner (FIG. 11C) and also substantially reduced VEGF-levels (FIG. 11D).

These data identify both IL8 and VEGF as candidate biomarkers that could be useful for evaluating the pharmacological activity of IL-1 pathway inhibitors in vivo. Similar to the data from FIGS. 9 and 10 this data is considered preliminary as it has not been proven in repetitive experiments.

### Example 9

### The IL1RA Drug Kineret (Amgen) Inhibits the Growth of CSC-Derived Xenograft Tumors & Modulates Serum Cytokines In Vivo

Based on the inventors' observations that an anti-IL1R1 antibody inhibit tumor-sphere formation in vitro it was further tested the inhibition of the IL-1 pathway in an in vivo CSC-xenograft model. The most appropriate and readily available molecule for such in vivo studies was the human IL1R-antagonist (IL1RA) which is known to cross-reach with mIL1R1. In this setting one can expect anti-tumor activity to be related to effects of IL1R1-inhibition on the tumor microenvironment (inflammatory infiltrating cells, stromal fibroblasts, etc.) as well as the CSC compartment. IL1RA is marketed under the trade name Kineret and the patent is held by Amgen (WO1989/011540). Currently, Kineret is approved for the treatment of rheumatoid arthritis.

Human cell cultures derived from primary tumors obtained from a CRC (FIG. 12A) and a NSCLC (FIG. 12B) patient were sorted for the Aldeflour-positive population using fluorescence activated cell sorting (FACS). Aldefluor is a marker of high aldehyde-dehydrogenase activity, which is associated with both normal stem cell and CSC phenotypes. The sorted aldefluor-positive (AF+) cells were suspended in Matrigel and injected subcutaneously at a very low cell number (2×10⁴ cells/mouse) into NOD/SCID immune-deficient mice. The matrigel suspensions contained a Kineret loading dose of 5 μg/mL which corresponds to reported serum levels of Kineret after daily s.c. dosing. Starting 1 day after inoculation of the AF+ cells, mice were treated daily with 5 mg or 10 mg of Kineret (purchased from Biovitrum) or the drug vehicle. The administration route was s.c. as is standard for Kineret in the clinic.

Tumor volume was monitored weekly for 76 days in the CRC model and for 91 days in the NSCLC model. In both models we observed a dose-dependent inhibition of tumor growth as can be seen in FIG. 12. In CRC we saw a strong inhibition at both the 5 and 10 mg doses (FIG. 12A), whereas for NSCLC response was only achieved with the higher dose of 10 mg/kg. Both doses were well tolerated by the mice.

Three mice from each study group were sacrificed at day 76 (CRC) or day 91 (NSCLC). The tumors were excised, dissociated into single cells, and viably cryopreserved. Serum was also collected for cytokine detection. For the remaining mice Kineret treatment was stopped and the mice were further monitored for tumor growth. When tumors reached ethical tumor-burden limits the mice were sacrificed and tumor cells and serum were collected. After Kineret treatment was stopped, there was one mouse in the CRC study that showed almost no progression and was designated as a high responder to Kineret (high responder), one mouse with a slow-growing tumor was designated an intermediate responder (medium responder) and one mouse with a rapidly-growing tumor was designated a low responder (low responder). From all sacrificed mice we also collected and cryopreserved bone marrow cells from the femur and tibial bones for possible analyses.

Serum cytokine analysis via ELISA revealed readily detectable levels of human IL8 and human VEGF in the vehicle controls groups of both the CRC and NSCLC models (FIG. 13). Treatment with Kineret did not alter hVEGF levels in mouse serum (FIG. 13C); however, hl L-8 was found to be reduced to undetectable levels (FIG. 13A).

As the tumors in the treated groups were inhibited in their growth it must be considered that reduced hIL8 levels are a direct reflection of reduced tumor size and therefore hIL-8 levels cannot be validated as a pharmacodynamic response biomarker in xenograft models (FIG. 13A). In the three different classifications of responders in the CRC model we again observed that amounts of hIL8 corresponded closely with tumor size (FIG. 13B) and we saw no changes in VEGF levels (FIG. 13D).

These preliminary pharmacology studies demonstrate that IL1R1 inhibition is efficacious in vivo against CSC-derived xenograft models of CRC and NSCLC. is Human IL8 levels in murine serum can be used as a disease response biomarker in these models correlating to tumor size and this will be useful in the future as a surrogate read-out for tumor burden in orthotopic models in which tumor volumes cannot be directly measured. Human VEGF does not hold value as an in vivo biomarker in these models as IL1R1 inhibition had no effect on hVEGF serum levels.

### Example 10

### Tumor Associated Macrophages (TAMS) Promote Tumor-Sphere Formation In Vitro Through a Mechanism Involving IL-1

Tumors are heavily infiltrated by so-called tumor-associated macrophages (TAM) that have been shown to promote tumor cell proliferation, tumor immune evasion, metastasis, and angiogenesis. Macrophages are the primary source of secreted IL-1 during inflammatory responses; taking this fact together with our findings of a role for IL-1 in supporting CSC the inventors hypothesize that macrophage-derived IL-1 may represent a key factor supporting the CSC-niche within tumors.

To experimentally address this hypothesis the above described and developed Her2/neu tumor-sphere/TAM co-culture model was used. Her2/neu mice are transgenic for the rat Her2/neu oncogene under transcriptional control of the mouse mammary tumor virus (MMTV) promoter. Her2/neu mice spontaneously develop breast tumors at ˜4 months of age. Methods were established for isolating TAM from Her2/neu tumors which involve excision of primary breast tumors, enzymatic dissociation of the tumors into single-cells, and depletion of fibroblast, endothelial and erythrocyte lineages by magnetic bead separation. The tumor cells and TAM are then sorted into separate populations via FACS. Finally, the TAM are placed together with the Her2/neu tumor cells under tumor-sphere promoting culture conditions (FIG. 14A).

TAM in culture cannot form spheroid structures, whereas HER2/neu tumor cells readily form breast tumor mammospheres (FIG. 14B). In multiple rounds of co-culture experiments we have shown that the presence of TAM consistently promotes HER2/neu-tumor-sphere formation. Furthermore, tumor-sphere numbers increase linearly in relation to increased numbers of co-plated TAMS (FIG. 14B).

To investigate the possible role of IL-1 signalling in TAM-supported tumor-sphere growth, HER2/neu-TAM co-cultures were treated with an anti-murine IL1R1 Mab at two different doses. IL1R1 blockade had no significant effect on cultures that contained only TAMS or only HER2/neu tumor cells. However, under co-culture conditions, IL1R1 blockade resulted in a clear dose-dependent reduction in Her2/neu tumor-sphere formation (FIG. 14C), supporting a role for the IL-1 pathway in TAM-mediated support of CSC.

Therefore it can be stated that IL1R1 inhibition has an effect on breast cancer stem cells and their protective microenvironment.

1-18. (canceled)

19. A polypeptide inhibiting the binding of IL1beta to IL1R1 and/or the heterodimerization of IL1R1 with IL1RaCP for use in the treatment of cancer cells and/or cancer stem cells (CSC) in an individual.

20. A polypeptide for use in the treatment of CSC according to claim 19.

21. A polypeptide for use in the treatment of CSC according to claim 20, wherein said CSC comprise other tumor cells, which are not CSC.

22. A polypeptide for use in the treatment of cancer cells according to claim 19, wherein said cancer cells further comprise CSC.

23. A polypeptide for use in the treatment of cancer cells and/or CSC according to claim 19, wherein IL1R is expressed on the surface of said CSC but not or not essentially on tumor cells which are not CSC.

24. A polypeptide for use in the treatment of cancer cells and/or CSC according to claim 19, wherein said cancer stem cells are resistant at least to chemotherapy and/or radiotherapy.

25. A polypeptide for use in the treatment of cancer cells and/or CSC according to claim 19, wherein the cancer to be treated is selected from the group consisting of: colorectal cancer (CRC), non small cell lung cancer (NSCLC) and breast cancer.

26. A polypeptide for use in the treatment of cancer cells and/or CSC according to claim 19, wherein the polypeptide is administered to the individual in combination with a cytostatic agent, a cytotoxic agent, or radiotherapy.

27. A polypeptide for use in the treatment of cancer cells and/or CSC according to claim 26, wherein the cytostatic or cytotoxic agent is an anti-tumor antibody or a chemotherapeutic agent.

28. A polypeptide for use in the treatment of cancer cells and/or CSC according to claim 26 or 27, wherein said polypeptide is applied to the individual prior to, simultaneously with, or after said cytostatic or said cytotoxic agent or said radiotherapy treatment.

29. A polypeptide for use in the treatment of cancer cells and/or CSC according to claim 19, wherein the polypeptide is a human, a chimeric, or a humanized monoclonal antibody.

30. A polypeptide for use in the treatment of cancer cells and/or CSC according to claim 29, wherein the monoclonal antibody is an anti-IL1R1 antibody, an anti-IL1beta antibody, an anti-IL1RaCP antibody, or a bispecific antibody targeting IL1R1 and IL1RaCP.

31. A polypeptide for use in the treatment of cancer cells and/or CSC according to claim 19, wherein the polypeptide is recombinant natural or modified IL1RA.

32. A polypeptide for use in the treatment of cancer cells and/or CSC according to claim 19, wherein the polypeptide is an IL1R1-IL1RaCP fusion protein.

33. A polypeptide for use in the treatment of cancer cells and/or CSC according to claim 19, wherein the polypeptide is component of a pharmaceutical composition comprising in a therapeutically effective amount said polypeptide together with a pharmaceutically acceptable excipient, diluent or carrier.

34. Use of a biomarker for assessing and predicting the effect of an agent on cancer in a cell based ex-vivo assay, wherein
(i) the biomarker is IL-1R1,
(ii) the cells are chemo- or radio-resistant cancer stem cells (CSC) expressing IL1R1 on their surface,
(ii) and said agent is a therapeutic polypeptide as specified in claim 19.

35. Use of a biomarker according to claim 34, wherein the CSC was obtained as subpopulation of cells of tumor tissue samples of an individual by treating these tissue samples with a chemotherapeutic agent or by radiation.

36. Use of a biomarker according to claim 34 or 35, wherein the cancer cells in said cell based assay derive from samples of an individual suffering from NSCLC, CRC or breast cancer.
